(19) United States
(12) Patent Application Publication
(10) Pub. No.: US 2003/0064434 A1
(43) Pub. Date: Apr. 3, 2003

(54) Novel G Protein-coupled receptor, GAVE1

(75) Inventors: Ali Ardati, (US); Kimberly Della Penna, (US); Asher Zilberstein, (US)

Correspondence Address: Dean H. Nakamura, Roylance Abrams Berdo & Goodman, Suite 600, 1300 19th Street, N.W., Washington, DC, 20036, US

(73) Assignee: Aventis Pharmaceuticals, Inc.

(21) Appl. No.: 09/863,455

(22) Filed: May 24, 2001

(51) Int. Cl. G01N 33/53; C07H 21/04; A61K 31/685; C12P 21/02; C12N 5/06; C07K 14/705

(52) U.S. Cl. 435/69.1; 435/7.1; 435/325; 435/320.1; 530/350; 536/23.5; 530/388.22; 514/78

(57) ABSTRACT

Novel GAVE1 polypeptides, proteins and nucleic acid molecules are provided. In addition to isolated, full-length GAVE1 proteins, isolated GAVE1 fusion proteins, antigenic peptides and anti-GAVE1 antibodies are provided. Moreover, GAVE1 nucleic acid molecules, recombinant expression vectors containing a nucleic acid encoding GAVE1, host cells into which the expression vectors have been introduced and nonhuman transgenic animals in which a GAVE1 gene has been introduced or disrupted are provided. Diagnostic, screening and therapeutic methods utilizing GAVE1 compositions or composition that detect GAVE1 also are provided. Methods for identifying GAVE1 agonists, antagonists, reverse agonists and the like are described.

### Background of the Invention

The G protein-coupled receptors (GPCRs) are a large family of integral membrane proteins that are responsible for cellular signal transduction. GPCRs respond to a variety of extracellular signals, including neurotransmitters, hormones, odorants and light and are capable of transducing signals across the cell membrane so as to initiate a second messenger response within the cell. Many therapeutic drugs in use today target GPCRs because the receptors mediate a wide variety of physiological responses, including vasodilation, heart rate, bronchodilation, endocrine secretion, and gut peristalsis.

GPCRs are characterized by an extracellular ligand binding domain, seven transmembrane domains and an intracellular domain that interacts with cellular elements associated with signaling. The functions the receptors perform, such as binding ligands and G proteins, are associated with the presence of certain amino acids in critical positions.

Molecular cloning identified a subfamily of GPCRs known as the endothelial differentiation genes (Edg). Edg GPCRs show amino acid sequence identity of 31% to 34% as a subfamily, but contain two homology clusters with greater similarity of structure and function. One homology cluster includes Edg-2, Edg-4, Edg-7 and Edg-8 proteins, that bind lysophosphatidic acid (LPA) but not sphingolipids. A second homology cluster encompasses Edg-1, Edg-3 and Edg-5 that bind sphingosine-1 -phosphate (S1P or SPP) but not LPA (Goetzl et al., Adv Exp Med Biol (1999) 469:259-264).

S1P is a potent, extracellular lysolipid phosphoric acid mediator that is released, for example, during platelet activation (Moser et al., J Cell Biol (1992) 116:1517-1526). S1P elicits a wide variety of responses by cells. Prominent among those are cell proliferation (Zhang et al., J Cell Biol (1991) 114:155-167; Bornfeldt et al., J Cell Biol (1995) 130:193-206; and Berger et al., Mol Pharmacol (1996) 50:451-457) and anti-apoptosis (Cuvillier et al., Nature (1996) 381:800-803; Edsall et al., J Neurosci (1997) 17:6952-6960).

A variety of studies have shown that differences in amino acid sequence in GPCRs account for differences in affinities towards either a natural ligand or a small molecule agonist or antagonist. In other words, minor differences in sequence can account for different binding affinities and activities. (See for example Meng et al., J Bio Chem (1996) 271(50):32016-20; Burd et al., J Bio Chem (1998) 273(51):34488-95; and Hurley et al., J Neurochem (1999) 72(l):413-21). In particular, studies have shown that amino acid sequence differences in the third intracellular domain can result in different activities. Myburgh et al. studied gonadotropin releasing hormone receptor, and demonstrated that alanine 261 in intracellular loop III of the receptor is crucial for G protein coupling and receptor internalization (Biochem J (1998) 331(Part 3):893-6). Wonerow et al. studied the thyrotropin receptor and demonstrated that deletions in the third intracellular loop resulted in constitutitive receptor activity (J Bio Chem (1998) 273(14):7900-5). That was the first report demonstrating that amino acid deletions within the third intracellular loop of a G protein-coupled receptor resulted in constitutive receptor activity.

Given the role GPCRs have in disease, and the ability to treat disease by modulating the activity of GPCRs, identification and characterization of previously unknown GPCRs can provide new compositions and methods of treatment for disease states that involve the activity of a GPCR. The instant invention identifies and characterizes the expression of a novel GPCR, GAVE1, and provides compositions and methods for applying the discovery to the identification and treatment of related diseases.

### SUMMARY OF THE INVENTION

The instant invention relates to a newly identified G Protein-coupled receptor. In particular, the instant G-coupled receptor is modulated by the alpha-adrenergic agonist, phenylephrine, in cardiomyocytes and is down regulated in T helper cells when activated (e.g., associated with inflammation). That and other information intimates that the novel receptor, identified as GAVE1, is involved in a variety of diseases, including ischemic heart failure, ischemic reperfusion injury, restenosis, dilated cardiomyopathy, apoptosis, such as cardiomyocyte apoptosis, atherosclerosis, stroke and various perturbations of the immune system.

In one aspect, the invention relates to isolated nucleic acids selected from the group consisting of an isolated nucleic acid which encodes a vertebrate protein of amino acids as set forth in SEQ ID NO: 2, variants, mutations and fragments thereof, and an isolated nucleic acid which comprises a nucleotide sequence as set forth in SEQ ID NO: 1, variants, mutations and fragments thereof. Further, the invention relates to nucleic acid hybridization probes and complementary fragments which bind to SEQ ID NO: 1 or hybridization probes and complementary fragments which bind to nucleic acids which encode the amino acid sequence as set forth in SEQ ID NO: 2. Further, the invention relates to nucleic acids having about 65%-99% identity to SEQ ID NO: 1, including nucleic acids having about 65%-99% identity to isolated nucleic acids encoding an amino acid sequence as set forth in SEQ ID NO: 2. In a related aspect, the oligonucleotides comprise at least 8 nucleotides and methods of hybridizing are contemplated comprising the steps of contacting the complementary oligonucleotide with a nucleic acid comprising the nucleotides as set forth in SEQ ID NO: 1 under conditions that permit hybridization of the complement with the nucleic acid. Further, complementary fragments may serve as anti-sense oligonucleotides for methods of inhibiting the expression of GAVE1, in vivo and in vitro. Such methods may comprise the steps of providing an oligonucleotide sequence consisting of the complement of the nucleotides as set forth in SEQ ID NO: 1, providing a human cell comprising an mRNA compromising the sequence of nucleotides as set forth in SEQ ID NO: 1 and introducing the oligonucleotide into the cell, where the expression of GAVE1 is inhibited by mechanisms which include inhibition of translation, triple helix formation and/or nuclease activation leading to degradation of mRNA in the cell.

The invention also relates to isolated polypeptides selected from the group consisting of purified polypeptides of amino acid sequence as set forth in SEQ ID NO: 2, variants, mutations and fragments thereof, and purified polypeptides having additional amino acid residues which provide functional properties to the polypeptide.

The invention further relates to the nucleic acids operably linked to expression control elements, including vectors comprising the isolated nucleic acids. The invention further relates to cultured cells transformed to comprise the nucleic acids of the invention. The invention includes methods for producing a polypeptide comprising the steps of growing transformed cells comprising the nucleic acids of the invention, permitting expression and purifying the polypeptide from the cell or medium in which a cell was cultured.

A further aspect of the invention includes an isolated antibody that binds to a polypeptide of the invention, including monoclonal and polyclonal antibodies. Further, in a related aspect, methods of producing antibodies and methods for treating GAVE1 related diseases with an antibody that binds to GAVE1 are disclosed.

An additional aspect of the invention includes methods, for diagnostic purposes, for determining the presence or absence of GAVE1 in a biological and/or tissue sample.

In another aspect of the invention, therapeutic methods are disclosed for modulating GAVE1 signal transduction, including administration of peptides, agonists, antagonists, inverse agonists and/or antibody to a patient in need thereof.

In another aspect of the invention, methods are disclosed for identifying modulators of GAVE1 comprising the steps of providing a chemical moiety, providing a cell expressing GAVE1 and determining whether the chemical moiety modulates the signaling activity of GAVE1. The method also includes identifying whether such modulation occurs in the presence or absence of an endogenous ligand. In a related aspect, the chemical moieties can include, but are not limited to, peptides, antibodies, agonists, inverse agonists and antagonists.

Another aspect of the invention includes therapeutic compositions, where such compositions include nucleic acids, antibodies, polypeptides, agonists, inverse agonists and antagonists. Further, methods of the invention also include methods of treating disease states and modulating GAVE1 signaling activity by administering such therapeutic compositions to a patient in need thereof.

Those and other aspects of the invention will become evident on reference to the following detailed description and the attached drawings. In addition, various references are set forth below which describe in more detail certain procedures or compositions. Each of those references is hereby incorporated herein by reference in entirety as if each were individually noted for incorporation.

### BRIEF DESCRIPTION OF THE DRAWINGS

FIG. 1 provides a nucleic acid sequence of GAVE1 (SEQ ID NO: 1).

FIG. 2 depicts an amino acid sequence of GAVE1 (SEQ ID NO: 2) Amino acid resides 250-252 are highlighted.

FIG. 3 is a computerized representation of a Northern blot of RNA obtained from various human tissues.

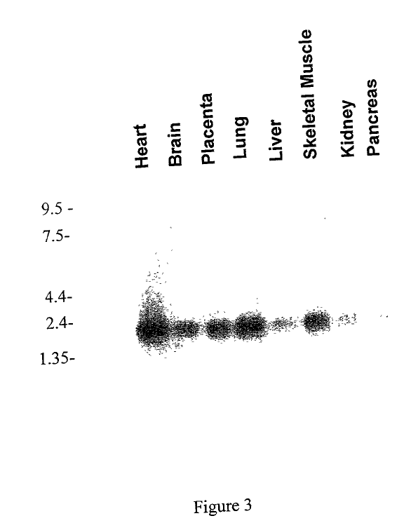

FIG. 4 depicts data obtained from Taqman assays demonstrating presence of GAVE1 RNA in samples obtained from a variety of human tissues.

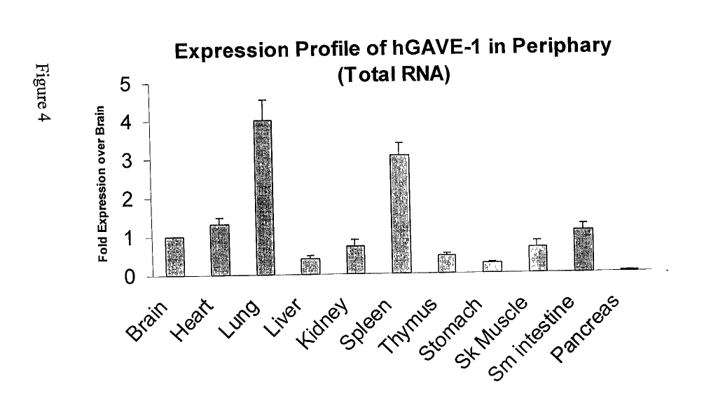

FIGS. 5A and 5B depict the results of Taqman assays on the RNA of various public cell lines obtained from the ATCC. Huvec are human umbilical endothelial cells, two different cell lines were tested. HAEC are human aortic endothelial cells. HCAEC are human coronary artery endothelial cells. HCACSM are human coronary artery smooth muscle cells. HSK muscle are human skeletal muscle cells.

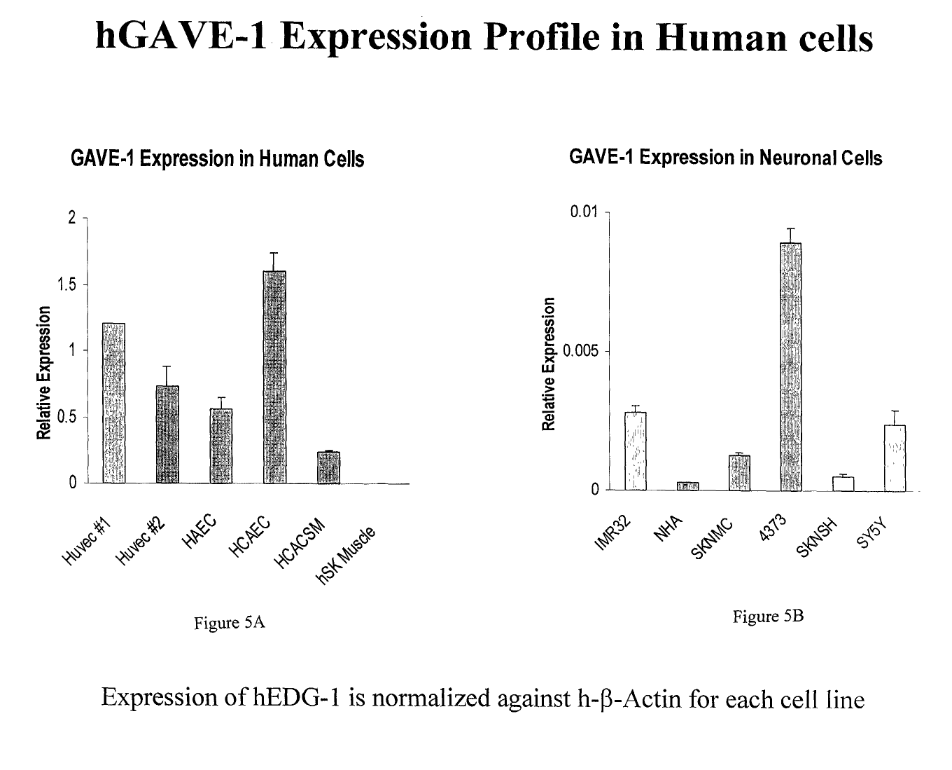

FIG. 6 depicts the results in the change in fluorescence using GAVE1 expressing cells stimulated with S1P (SPP) in a FLIPR assay. CHO cells (curve denoted with diamonds, demonstrates a control level of GAVE1 activity with increasing amounts of ligand S-1-P. CHO cells expressing GAVE1 demonstrate a positive correlation of activity with increasing amounts of ligand.

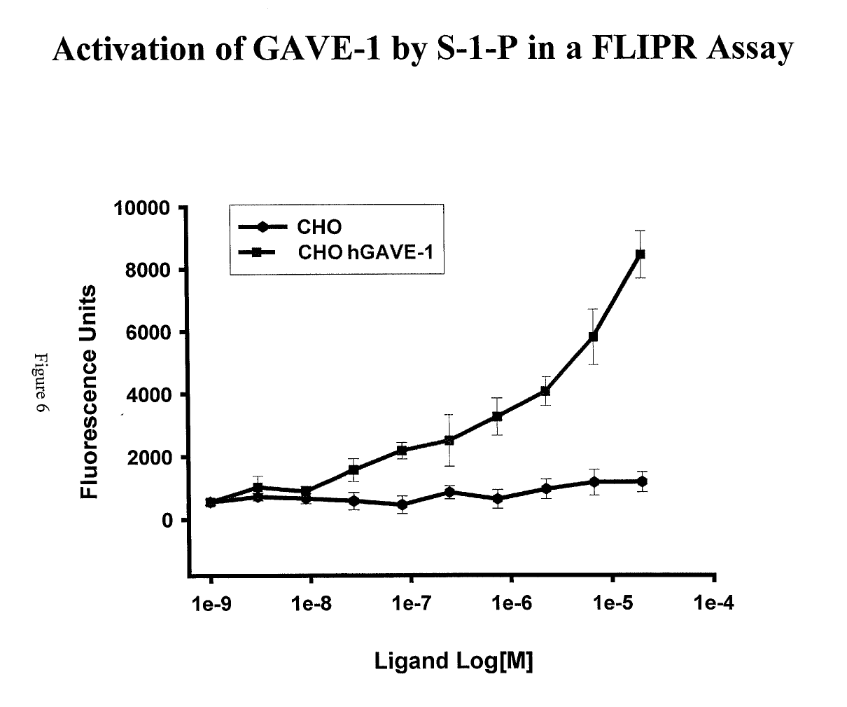

FIG. 7 is a computer representation of a Northern blot depicting GAVE1 RNA levels of cardiomyocytes expressing GAVE1 exposed to medium, vehicle and vehicle containing phenylephrine.

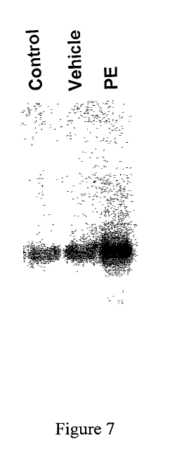

FIG. 8 depicts an assay of GAVE1 expression in Th1 and Th2 lymphocytes. The cells were exposed to CD3 and CD8 antibodies to activate the cells and tested for GAVE1 message. The activated cells were tested at two time points.

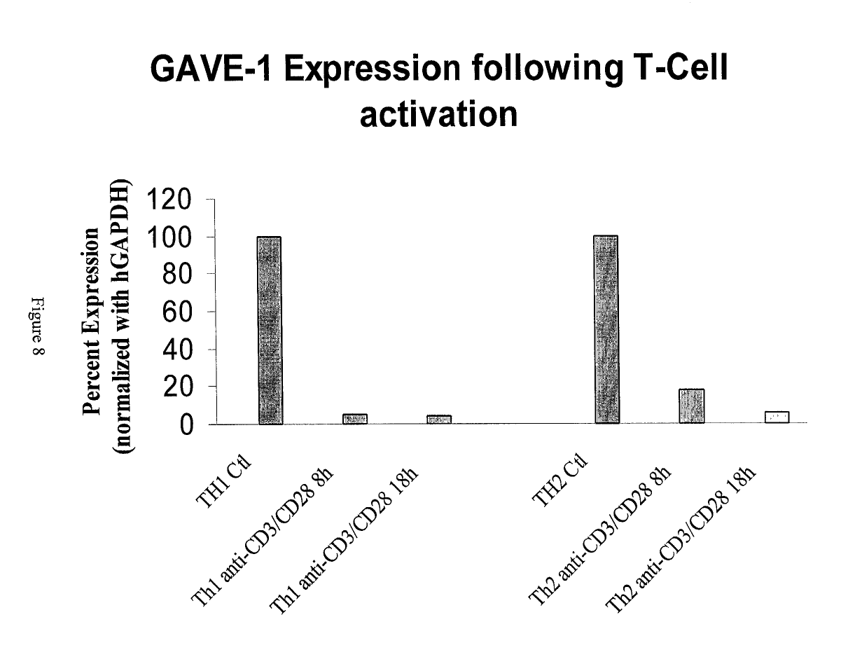

### DETAILED DESCRIPTION OF THE INVENTION

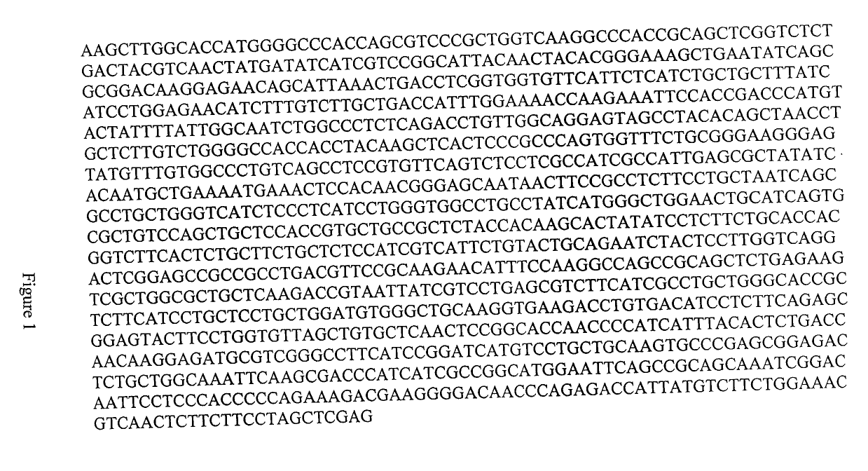

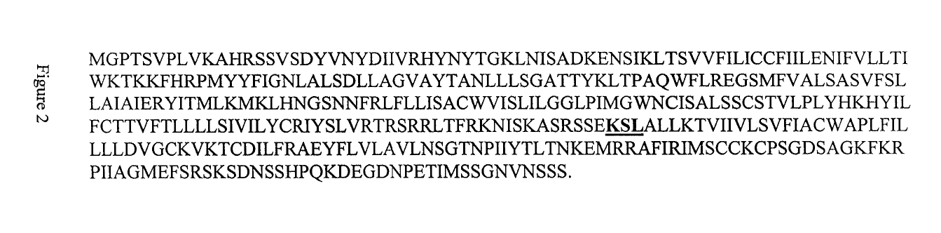

The instant invention is based on the discovery of a cDNA molecule encoding human GAVE1, a member of the G protein-coupled receptor superfamily, which binds to the endogenous ligand, sphingosine-1-phosphate (S1P, S-1-P, SPP-1 or SSP.) A nucleotide sequence encoding a human GAVE1 protein is shown in FIG. 1 (SEQ ID NO: 1). An amino acid sequence of GAVE1 protein is shown in FIG. 2 (SEQ ID NO: 2).

The GAVE1 cDNA of FIG. 1 (SEQ ID NO: 1), which is approximately 1170 nucleotides long, including untranslated regions encodes a protein having a molecular weight of approximately 42.8 kDa (excluding post-translational modifications).

Using a TaqMan® assay, a specific mRNA fragment is expressed at moderate levels in skeletal muscle, placenta, liver, kidney and intestine. Higher levels were observed in brain, heart, spleen and lung. Further, GAVE1 expression is induced selectively in cardiomyocytes in the presence of phenylephrine.

The presence of GAVE1 in those and other tissues suggests GAVE1 is involved in a variety of disease states, including ischemic heart failure, ischemic reperfusion injury, inflammation, dilated cardiomyopathy, apoptosis, including cardiac myocyte apoptosis, atherosclerosis, immune functions, stroke and various neurodegenerative diseases. Identification of GAVE1 in those tissues and cloning of the gene encoding GAVE1 provides a variety of therapeutic approaches to regulate GAVE1 expression and activity so as to provide therapeutic approaches to treating diseases involving GAVE1.

Human GAVE1 is related to the Edg family of molecules having certain conserved structural and functional features. The term “family,” when referring to the protein and nucleic acid molecules of the invention, is intended to mean two or more proteins or nucleic acid molecules having an overall common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family also may have common functional characteristics.

The Edg-1 receptor was isolated first by Hla & Maciag following stimulation of a human endothelial cell line by phorbol esters to identify genes implicated in cell differentiation (J Biol Chem (1990) 265:9308-9313). At that time, Edg-1 was classified as an orphan GPCR since no ligand was known to activate the receptor. Lee et al. later identified a serum-borne phospholipid called sphingosine-1-phosphate as the endogenous ligand for Edg-1 receptor (Science (1998) 279:1552-1555).

The distinctive tissue distribution of GAVE1 expression directed efforts to the cardiovascular system. Also, GAVE1 has a role in immune cell function. For example, down regulation of GAVE1 in immune cells is observed frequently.

The receptor also is conserved and is found in, for example, human, mouse and rat. Certain domains are well-conserved across species.

GAVE1 also demonstrated modulation of expression associated with apoptosis. Thus, GAVE1 can be manipulated to induce or curtail programmed cell death.

In one embodiment, a GAVE1 protein includes a third intracellular loop, domain, about amino acids 230 to about 265, having at least about 65%, preferably at least about 75%, and more preferably about 85%, 95% or 98% amino acid sequence identity to the third intracellular loop domain of SEQ ID NO: 2, wherein equivalent amino acid residues at positions 250, 251 and 252 are invariant. In a related aspect, a GAVE1 protein includes the carboxyl terminal domain having at least about 65%, preferably at least about 75%, and more preferably about 85%, 95% or 98% amino acid sequence identity to the carboxyl terminal domain of SEQ ID NO: 2, wherein an equivalent amino acid residue at position 382 is invariant. The term “equivalent amino acid residues” herein means the amino acids occupy substantially the same position within a protein sequence when two or more sequences are aligned for analysis. Preferred GAVE1 polypeptides of the instant invention have an amino acid sequence sufficiently identical to the third intracellular loop domain consensus amino acid sequence of SEQ ID NO: 2. The term “sufficiently identical” is used herein to refer to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95% or 98% identity are defined herein as sufficiently identical.

Other domains of interest include, but are not limited to, the transmembrane (TM) domains (TM1 from about amino acid residue 55 to about 75; TM2 from about amino acid residue 85 to about 110; TM3 from about amino acid residue 125 to about 150; TM4 from about amino acid residue 165 to about 190; TM5 from about amino acid residue 205 to about 230; TM6 from about 260 to about 285; and TM7 from about amino acid residue 300 to about 320 as set forth in SEQ ID NO: 2); cytoplasmic (intracellular loop) domains (from about amino acid residue 75 to about 85; from about amino acid residue 150 to about 170; from about amino acid residue 230 to about 265; and from about amino acid residue 320 to about 390 as set forth in SEQ ID NO: 2); and extracellular domains (from about amino acid residue 1 to about 55; from about amino acid residue 110 to about 125; from about amino acid 190 to about 210; and from about amino acid residue 285 to about 300 as set forth in SEQ ID NO: 2). In a related aspect, domains of interest also include, but are not limited to, consensus glycosylation sites, lipid binding sites and phosphorylation sites.

As used interchangeably herein, a “GAVE1 activity”, “biological activity of GAVE” or “functional activity of GAVE1”, refers to an activity exerted by a GAVE1 protein, polypeptide or nucleic acid molecule on a GAVE1 responsive cell as determined in vivo or in vitro, according to standard techniques. A GAVE1 activity can be a direct activity, such as an association with or an enzymatic activity on a second protein, or an indirect activity, such as a cellular signaling activity mediated by interaction of the GAVE1 protein with a second protein. In a preferred embodiment, a GAVE1 activity includes at least one or more of the following activities: (i) the ability to interact with proteins in the GAVE1 signaling pathway; (ii) the ability to interact with a GAVE1 ligand; and (iii) the ability to interact with an intracellular target protein. For example, a GAVE1 activity includes, but is not limited to, induction of GAVE1 specific mRNA by phenylephrine. In another embodiment, a GAVE1 activity includes, but is not limited to, binding of sphingosine-1-phosphate (S-1-P), as may be determined by means such as fluorescent change (by FLIPR® analysis) or binding of labeled S-1-P in cells transformed with an expression vector comprising SEQ ID NO: 1.

Accordingly, another embodiment of the invention features isolated GAVE1 proteins and polypeptides having a GAVE1 activity.

Various aspects of the invention are described in further detail in the following subsections.

### I. Isolated Nucleic Acid Molecules

One aspect of the invention pertains to isolated nucleic acid molecules that encode GAVE1 proteins or biologically active portions thereof, as well as nucleic acid molecules sufficient for use as hybridization probes to identify GAVE1-encoding nucleic acids (e.g., GAVE1 mRNA) and fragments for use as PCR primers for the amplification or mutation of GAVE1 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded.

An “isolated” nucleic acid molecule is one that is separated from other nucleic acid molecules that are present in the natural source of the nucleic acid. Preferably, an “isolated” nucleic acid is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated GAVE1 nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the instant invention, e.g., a nucleic acid molecule having the nucleotide sequence of SEQ ID NO: 1, or a complement of any of those nucleotide sequences, can be isolated using standard molecular biology techniques and the sequence information provided herein. Using all or a portion of the nucleic acid sequences of SEQ ID NO: 1 as a hybridization probe, GAVE1 nucleic acid molecules can be isolated using standard hybridization and cloning techniques (e.g., as described in Sambrook et al., eds., “Molecular Cloning: A Laboratory Manual,” 2nd ed., Cold Spring Harbor Laboratory Press, Cold Spring Harbor, N.Y., 1989).

A nucleic acid molecule of the invention, or portion thereof, can be amplified using cDNA, mRNA or genomic DNA as a template and appropriate oligonucleotide primers according to standard PCR amplification techniques. For example, such primers can comprise, but are not limited to 5′-TCTGCGGGAAGGGAGTATGT-3′ (SEQ ID NO: 3) and 5′-CGATGGCGAGGAGACTGAA-3′ (SEQ ID NO: 4). The nucleic acid so amplified can be cloned into an appropriate vector and characterized by DNA sequence analysis. Furthermore, oligonucleotides corresponding to GAVE1 nucleotide sequences can be prepared by standard synthetic techniques, e.g., using an automated DNA synthesizer.

In another preferred embodiment, an isolated nucleic acid molecule of the invention comprises a nucleic acid molecule that is a complement of the nucleotide sequence shown in SEQ ID NO: 1, or a portion thereof. A nucleic acid molecule which is complementary to a given nucleotide sequence is one which is sufficiently complementary to the given nucleotide sequence that it can hybridize to the given nucleotide sequence to thereby form a stable duplex.

Moreover, the nucleic acid molecule of the invention can comprise only a portion of a nucleic acid sequence encoding GAVE1, for example, a fragment that can be used as a probe or primer or a fragment encoding a biologically active portion of GAVE1. For example, such a fragment can comprise, but is not limited to, a region encoding amino acid residues 250-252 as set forth in SEQ ID NO: 2. Further, such fragments may comprise a region encoding the amino acid residue 382 as set forth in SEQ ID NO: 2. The nucleotide sequence determined from the cloning of the human GAVE1 gene allows for the generation of probes and primers designed for use in identifying and/or cloning GAVE1 homologues in other cell types, e.g., from other tissues, as well as GAVE1 homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of SEQ ID NO: 1 or of a naturally occurring mutant of SEQ ID NO: 1. Probes based on the human GAVE1 nucleotide sequence can be used to detect transcripts or genomic sequences encoding the similar or identical proteins. The probe may comprise a label group attached thereto, e.g., a radioisotope, a fluorescent compound, an enzyme or an enzyme co-factor. Such probes can be used as part of a diagnostic test kit for identifying cells or tissues which improperly express a GAVE1 protein, such as by measuring levels of a GAVE1 -encoding nucleic acid in a sample of cells from a subject, e.g., detecting GAVE1 mRNA levels or determining whether a genomic GAVE1 gene has been mutated or deleted.

A nucleic acid fragment encoding a “biologically active portion of GAVE1” can be prepared by isolating a portion of SEQ ID NO: 1 which encodes a polypeptide having a GAVE1 biological activity, expressing the encoded portion of GAVE1 protein (e.g., by recombinant expression in vitro) and assessing the activity of the encoded portion of GAVE1. For example, a nucleic acid fragment encoding a biologically active portion of GAVE1 includes a third intracellular loop domain, e.g., amino acid residues from about 223 to about 255 as set forth in SEQ ID NO: 2. The invention further encompasses nucleic acid molecules that differ from the nucleotide sequence of SEQ ID NO: 1 due to degeneracy of the genetic code and thus encode the same GAVE1 protein as that encoded by the nucleotide sequence shown in SEQ ID NO: 1.

In addition to the human GAVE1 nucleotide sequence shown in SEQ ID NO: 1, it will be appreciated by those skilled in the art that DNA sequence polymorphisms that lead to changes in the amino acid sequences of GAVE1 may exist within a population (e.g., the human population). Such genetic polymorphism in the GAVE1 gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a GAVE1 protein, preferably a mammalian GAVE1 protein. As used herein, the phrase “allelic variant” refers to a nucleotide sequence that occurs at a GAVE1 locus or to a polypeptide encoded by the nucleotide sequence. Alternative alleles can be identified by sequencing the gene of interest in a number of different individuals. That can be carried out readily by using hybridization probes to identify the same genetic locus in a variety of individuals. Any and all such nucleotide variations and resulting amino acid polymorphisms or variations in GAVE1 that are the result of natural allelic variation and that do not alter the functional activity of GAVE1 are intended to be within the scope of the invention.

Moreover, nucleic acid molecules encoding GAVE1 proteins from other species (GAVE1 homologues), which have a nucleotide sequence which differs from that of a human GAVE1, are intended to be within the scope of the invention. Nucleic acid molecules corresponding to natural allelic variants and homologues of the GAVE1 cDNA of the invention can be isolated based on identity to the human GAVE1 nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 300, 325, 350, 375, 400, 425, 450, 500, 550, 600, 650, 700, 800, 900, 1000 or 1100 nucleotides in length and hybridizes under stringent conditions to the nucleic acid molecule comprising the nucleotide sequence, preferably the coding sequence of SEQ ID NO: 1, or a complement thereof.

As used herein, the term “hybridizes under stringent conditions” is intended to describe conditions for hybridization and washing under which nucleotide sequences at least 60% (65%, 70% preferably 75% or greater) identical to each other typically remain hybridized to each other. Such stringent conditions are known to those skilled in the art and can be found, for example, in “Current Protocols in Molecular Biology,” John Wiley & Sons, N.Y. (1989), 6.3.1-6.3.6. A preferred, non-limiting example of stringent hybridization conditions is hybridization in 6× sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions to the sequence of SEQ ID NO: 1 or the complement thereof corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the GAVE1 sequence that may exist in the population, the skilled artisan will further appreciate that changes can be introduced by mutation into the nucleotide sequence of SEQ ID NO: 1, thereby leading to changes in the amino acid sequence of the encoded GAVE1 protein, without altering the biological activity of the GAVE1 protein. For example, one can make nucleotide substitutions leading to amino acid substitutions at “non-essential” amino acid residues. A “non-essential” amino acid residue is a residue that can be altered from the wild-type sequence of GAVE1 (e.g., the sequence of SEQ ID NO: 2) without altering the biological activity, whereas an “essential” amino acid residue is required for biological activity. For example, amino acid residues that are not conserved or only semi-conserved among GAVE1 of various species may be non-essential for activity and thus would be likely targets for alteration. Alternatively, amino acid residues that are conserved among the GAVE1 proteins of various species may be essential for activity and thus would not be likely targets for alteration. For example, sequences comprising mutations that do not alter residues 250-252 and/or residue 382, as set forth in SEQ ID NO: 2 also may be included as (but not limited to) substitutions which alter the wild-type sequence.

Accordingly, another aspect of the invention pertains to nucleic acid molecules encoding GAVE1 proteins that contain changes in amino acid residues that are not essential for activity. Such GAVE1 proteins differ in amino acid sequence from SEQ ID NO: 2 yet retain biological activity. In one embodiment, the isolated nucleic acid molecule includes a nucleotide sequence encoding a protein that includes an amino acid sequence that is at least about 45% identical, 65%, 75%, 85%, 95%, 98% or 99% identical to the amino acid sequence of SEQ ID NO: 2.

An isolated nucleic acid molecule encoding a GAVE1 protein having a sequence which differs from that of SEQ ID NO: 2 can be created by introducing one or more nucleotide substitutions, additions or deletions into the nucleotide sequence of SEQ ID NO: 1 such that: 1) one or more amino acid substitutions, additions or deletions are introduced into the encoded protein and 2) residues 250-252 and/or residue 382 of SEQ ID NO: 2 remain invariant.

Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Those families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in GAVE1 is preferably replaced with another amino acid residue from the same side chain family. Alternatively, mutations can be introduced randomly along all or part of a GAVE1 coding sequence, such as by saturation mutagenesis, and the resultant mutants can be screened for GAVE1 biological activity to identify mutants that retain activity. Following mutagenesis, the encoded protein can be expressed recombinantly and the activity of the protein can be determined.

In a preferred embodiment, a mutant GAVE1 protein can be assayed for: (1) the ability to form protein:protein interactions with proteins in the GAVE1 signaling pathway; (2) the ability to bind a GAVE1 ligand (e.g., S1P); or (3) the ability to bind to an intracellular target protein. In yet another preferred embodiment, a mutant GAVE1 can be assayed for the ability to modulate cellular proliferation or cellular differentiation.

The instant invention encompasses antisense nucleic acid molecules, i.e., molecules which are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire GAVE1 coding strand, or to only a portion thereof, e.g., all or part of the protein coding region (or open reading frame). An antisense nucleic acid molecule can be antisense to a noncoding region of the coding strand of a nucleotide sequence encoding GAVE1. The noncoding regions (“5′ and 3′ untranslated or flanking regions”) are the 5′ and 3′ sequences that flank the coding region and are not translated into amino acids.

Given the coding strand sequences encoding GAVE1 disclosed herein (e.g., SEQ ID NO: 1), antisense nucleic acids of the invention can be designed according to the rules of Watson and Crick base pairing. The antisense nucleic acid molecule can be complementary to the entire coding region of GAVE1 mRNA, but more preferably is an oligonucleotide that is antisense to only a portion of the coding or noncoding region of GAVE1 mRNA. For example, the antisense oligonucleotide can be complementary to the region near the translation start site of GAVE1 mRNA, such as an oligonucleotide having the sequence 5′-GTGGGCCCCATGGTGCCAAGCTT-3′ (SEQ ID NO: 5) or 5′-GAGCTAGGAAGAAGAGTT-3′ (SEQ ID NO: 6). Alternatively, the antisense molecule can be directed to regulatory regions associated with expression of GAVE1. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be synthesized chemically using naturally occurring nucleotides or variously modified nucleotides designed to increase the biological stability of the molecules or to increase the physical stability of the duplex formed between the antisense and sense nucleic acids, e.g., phosphorothioate derivatives, phosphonate derivatives and acridine substituted nucleotides can be used.

Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, β-D-mannosylqueosine, 5-methoxycarboxymethyluracil, 5-methoxyuracil, 2-methylthio-N⁶-isopentenyladenine, uracil-5-oxyacetic acid, butoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3 -amino-3-N-2-carboxypropyl) uracil and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a GAVE1 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracelluar concentrations of the antisense molecules, vector constructs in which the antisense nucleic acid molecule is placed under the control of a strong RNA polymerase (pol) II or pol III promoter are preferred.

An antisense nucleic acid molecule of the invention can be an a-anomeric nucleic acid molecule. An α-anomeric nucleic acid molecule forms specific double-stranded hybrids with complementary RNA in which the strands run parallel to each other (Gaultier et al., Nucleic Acids Res (1987)15:6625-6641). The antisense nucleic acid molecule can also comprise a methylribonucleotide (Inoue et al., (1987) Nucleic Acids Res 15:6131-6148) or a chimeric RNA-DNA analogue (Inoue et al., (1987) FEBS Lett 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff et al., Nature (1988) 334:585-591)) can be used to catalytically cleave GAVE1 mRNA transcripts to thereby inhibit translation of GAVE1 mRNA. A ribozyme having specificity for a GAVE1-encoding nucleic acid can be designed based on the nucleotide sequence of a GAVE1 cDNA disclosed herein (e.g., SEQ ID NO: 1). For example, a derivative of a Tetrahymena L-19 IVS RNA can be constructed in which the nucleotide sequence of the active site is complementary to the nucleotide sequence to be cleaved in a GAVE1-encoding mRNA. See, e.g., Cech et al., U.S. Pat. No. 4,987,071; and Cech et al., U.S. Pat. No. 5,116,742. Alternatively, GAVE1 mRNA can be used to select a catalytic RNA having a specific ribonuclease activity from a pool of RNA molecules. See, e.g., Bartel et al., Science (1993) 261:1411-1418.

The invention also encompasses nucleic acid molecules that form triple helical structures. For example, GAVE1 gene expression can be inhibited by targeting nucleotide sequences complementary to the regulatory region of the GAVE1 (e.g., the GAVE1 promoter and/or enhancers) to form triple helical structures that prevent transcription of the GAVE1 gene in target cells. See generally Helene, Anticancer Drug Dis (1991) 6(6):569; Helene, Ann NY Acad Sci (1992) 660:27; and Maher, Bioassays (1992) 14(12):807.

In preferred embodiments, the nucleic acid molecules of the invention can be modified at the base moiety, sugar moiety or phosphate backbone to improve, e.g., the stability, hybridization or solubility of the molecule. For example, the deoxyribose phosphate backbone of the nucleic acids can be modified to generate peptide nucleic acids (See Hyrup et al., Bioorganic & Medicinal Chemistry (1996) 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-OKeefe et al., Proc Natl Acad Sci USA (1996) 93:14670.

PNAs of GAVE1 can be used in therapeutic and diagnostic applications. For example, PNAs can be used as antisense or antigene agents for sequence-specific modulation of gene expression by, e.g., inducing transcription or translation arrest or inhibiting replication. PNAs of GAVE1 can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra) or as probes or primers for DNA sequence and hybridization (Hyrup (1996), supra; Perry-O'Keefe et al. (1996), supra).

In another embodiment, PNAs of GAVE1 can be modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al., Nucleic Acids Res (1996) 24(17):3357-63, Mag et al., Nucleic Acids Res (1989) 17:5973; and Peterser et al., Bioorganic Med Chem Lett (1975) 5:1119.

### II. Isolated GAVE1 Proteins and Anti-GAVE1 Antibodies

One aspect of the invention pertains to isolated GAVE1 proteins, and biologically active portions thereof, as well as polypeptide fragments suitable, for example, for use as immunogens to raise anti-GAVE1 antibodies. In one embodiment, native GAVE1 proteins can be isolated from cells or tissue sources by an appropriate purification scheme using standard protein purification techniques. In another embodiment, GAVE1 proteins are produced by recombinant DNA techniques. Alternative to recombinant expression, a GAVE1 protein or polypeptide can be synthesized chemically using standard peptide synthesis techniques.

An “isolated” or “purified” protein or biologically active portion thereof is substantially free of cellular material or other contaminating proteins from the cell or tissue source from which the GAVE1 protein is derived, or substantially free of chemical precursors or other chemicals when chemically synthesized. The language “substantially free of cellular material” includes preparations of GAVE1 protein in which the protein is separated from cellular components of the cells from which it is isolated or recombinantly produced. Thus, GAVE1 protein that is substantially free of cellular material includes preparations of GAVE1 protein having less than about 30%, 20%, 10% or 5% (by dry weight) of non-GAVE1 protein (also referred to herein as a “contaminating protein”). When the GAVE1 protein or biologically active portion thereof is produced recombinantly, it is also preferably substantially free of culture medium, i.e., culture medium represents less than about 20%, 10% or 5% of the volume of the protein preparation. When GAVE1 protein is produced by chemical synthesis, it is preferably substantially free of chemical precursors or other chemicals, i.e., it is separated from chemical precursors or other chemicals which are involved in the synthesis of the protein. Accordingly, such preparations of GAVE1 protein have less than about 30%, 20%, 10% or 5% (by dry weight) of chemical precursors or non-GAVE1 chemicals.

Biologically active portions of a GAVE1 protein include peptides comprising amino acid sequences sufficiently identical to or derived from the amino acid sequence of the GAVE1 protein (e.g., the amino acid sequence shown in SEQ ID NO: 2), which include fewer amino acids than the full length GAVE1 proteins, and exhibit at least one activity of a GAVE1 protein. Typically, biologically active portions comprise a domain or motif with at least one activity of the GAVE1 protein. A biologically active portion of a GAVE1 protein can be a polypeptide that is, for example, 10, 25, 50, 100 or more amino acids in length. Preferred biologically active polypeptides include one or more identified GAVE1 structural domains, e.g., the third intracellular loop domain (e.g., SEQ ID NO: 2).

Moreover, other biologically active portions, in which other regions of the protein are deleted, can be prepared by recombinant techniques and evaluated for one or more of the functional activities of a native GAVE1 protein.

A preferred GAVE1 protein has the amino acid sequence shown of SEQ ID NO: 2. Other useful GAVE1 proteins are substantially identical to SEQ ID NO: 2 and retain the functional activity of the protein of SEQ ID NO: 2 yet differ in amino acid sequence due to natural allelic variation or mutagenesis. For example, such GAVE1 proteins and polypeptides possess at least one biological activity described herein.

Accordingly, a useful GAVE1 protein is a protein which includes an amino acid sequence at least about 45%, preferably 55%, 65%, 75%, 85%, 95% or 99% identical to the amino acid sequence of SEQ ID NO: 2 and retains the functional activity of the GAVE1 proteins of SEQ ID NO: 2. In other instances, the GAVE1 protein is a protein having an amino acid sequence 55%, 65%, 75%, 85%, 95% or 99% identical to the GAVE1 third intracellular loop domain (SEQ ID NO: 2). In a preferred embodiment, the GAVE1 protein retains a functional activity of the GAVE1 protein of SEQ ID NO: 2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions then are compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., percent identity=number of identical positions/total number of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin et al., Proc Natl Acad Sci USA (1990) 87:2264, modified as in Karlin et al., Proc Natl Acad Sci USA (1993) 90:5873-5877. Such an algorithm is incorporated into the NBLAST and XBLAST programs of Altschul et al., J Mol Bio (1990) 215:403. BLAST nucleotide searches can be performed with the NBLAST program, score=100, wordlength=12 to obtain nucleotide sequences homologous to GAVE1 nucleic acid molecules of the invention. BLAST protein searches can be performed with the XBLAST program, score=50, wordlength=3, to obtain amino acid sequences homologous to GAVE1 protein molecules of the invention. To obtain gapped alignments for comparison purposes, Gapped BLAST can be utilized as described in Altschul et al., Nucleic Acids Res (1997) 25:3389. Alternatively, PSI-Blast can be used to perform an iterated search that detects distant relationships between molecules. Altschul et al., (1997) supra. When utilizing BLAST, Gapped BLAST, and PSI-Blast programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers et al., CABIOS (1988) 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also provides GAVE1 chimeric or fusion proteins. As used herein, a GAVE1 “chimeric protein” or “fusion protein” comprises a GAVE1 polypeptide operably linked to a non-GAVE1 polypeptide. A “GAVE1 polypeptide” refers to a polypeptide having an amino acid sequence corresponding to GAVE1, whereas a “non-GAVE1 polypeptide” refers to a polypeptide having an amino acid sequence corresponding to a protein which is not substantially identical to the GAVE1 protein, e.g., a protein which is different from the GAVE1 protein and which is derived from the same or a different organism. Within a GAVE1 fusion protein the GAVE1 polypeptide can correspond to all or a portion of a GAVE1 protein, preferably at least one biologically active portion of a GAVE1 protein. Within the fusion protein, the term “operably linked” is intended to indicate that the GAVE1 polypeptide and the non-GAVE1 polypeptide are fused in-frame to each other. The non-GAVE1 polypeptide can be fused to the N-terminus or C-terminus of the GAVE1 polypeptide. One useful fusion protein is a GST-GAVE1 fusion protein in which the GAVE1 sequences are fused to the C-terminus of a glutathione-S-transferase (GST) sequence. Such fusion proteins can facilitate the purification of recombinant GAVE1.

In a preferred embodiment, the third intracellular loop (IL3) of the instant invention (i.e., from about 223 to about 255 as set forth in SEQ ID NO: 2) is fused with GST by PCR amplification of the IL3 and subcloning the product into a vector, such as, pGEX-2T. The resulting construct can be introduced into a host cell (e.g., E. coli) and expression from said construct can be induced by an appropriate small molecule (e.g., isopropyl-1-thio-β-D-galactopyranoside) and subsequently purified (See, e.g., Lee et al., J Biol Chem (1996) 271(19):11272-11279).

In certain host cells (e.g., mammalian host cells), expression and/or secretion of GAVE1 can be increased through use of a heterologous signal sequence. For example, the gp6® secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is a GAVE1-immunoglobulin fusion protein in which all or part of GAVE1 is fused to sequences derived from a member of the immunoglobulin protein family. The GAVE1-immunoglobulin fusion proteins of the invention can be incorporated into pharmaceutical compositions and administered to a subject to inhibit an interaction between a GAVE1 ligand and a GAVE1 protein on the surface of a cell, to thereby suppress GAVE1-mediated signal transduction in vivo. The GAVE1-immunoglobulin fusion proteins can be used to affect the bioavailability of a GAVE1 cognate ligand. Inhibition of the GAVE1 ligand-GAVE1 interaction may be useful therapeutically, both for treating proliferative and differentiative disorders and for modulating (e.g. promoting or inhibiting) cell survival. Moreover, the GAVE1-immunoglobulin fusion proteins of the invention can be used as immunogens to produce anti-GAVE1 antibodies in a subject, to purify GAVE1 ligands and in screening assays to identify molecules which inhibit the interaction of GAVE1 with a GAVE1 ligand.

Preferably, a GAVE1 chimeric or fusion protein of the invention is produced by standard recombinant DNA techniques. For example, DNA fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (See e.g., Ausubel et al., supra). Moreover, many expression vectors are commercially available that already encode a fusion moiety (e.g., a GST polypeptide). A GAVE1-encoding nucleic acid can be cloned into such an expression vector such that the fusion moiety is linked in-frame to the GAVE1 protein.

The instant invention also pertains to variants of the GAVE1 proteins (i.e., proteins having a sequence that differs from that of the GAVE1 amino acid sequence). Such variants can function as either GAVE1 agonists (mimetics) or as GAVE1 antagonists. Variants of the GAVE1 protein can be generated by mutagenesis, e.g., discrete point mutation or truncation of the GAVE1 protein. An agonist of the GAVE1 protein can retain substantially the same, or a subset, of the biological activities of the naturally occurring form of the GAVE1 protein. An antagonist of the GAVE1 protein can inhibit one or more of the activities of the naturally occurring form of the GAVE1 protein by, for example, competitively binding to a downstream or upstream member of a cellular signaling cascade which includes the GAVE1 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the GAVE1 proteins.

Variants of the GAVE1 protein which function as either GAVE1 agonists (mimetics) or as GAVE1 antagonists can be identified by screening combinatorial libraries of mutants, e.g., truncation mutants, of the GAVE1 protein for GAVE1 protein agonist or antagonist activity. In one embodiment, a variegated library of GAVE1 variants is generated by combinatorial mutagenesis at the nucleic acid level and is encoded by a variegated gene library. A variegated library of GAVE1 variants can be produced by, for example, enzymatically ligating a mixture of synthetic oligonucleotides into gene sequences such that a degenerate set of potential GAVE1 sequences is expressible as individual polypeptides, or alternatively, as a set of larger fusion proteins (e.g., for phage display) containing the set of GAVE1 sequences therein. There are a variety of methods that can be used to produce libraries of potential GAVE1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GAVE1 sequences. Methods for synthesizing degenerate oligonucleotides are known in the art (See, e.g., Narang, Tetrahedron (1983) 39:3; Itakura et al., Ann Rev Biochem (1984) 53:323; Itakura et al., Science (1984) 198:1056; Ike et al., Nucleic Acid Res (1983) 11:477).

In addition, libraries of fragments of the GAVE1 protein coding sequence can be used to generate a variegated population of GAVE1 fragments for screening and subsequent selection of variants of a GAVE1 protein. In one embodiment, a library of coding sequence fragments can be generated by treating a double-stranded PCR fragment of a GAVE1 coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By that method, an expression library can be derived which encodes N-terminal and internal fragments of various sizes of the GAVE1 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GAVE1 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify GAVE1 variants (Arkin et al., Proc Natl Acad Sci USA (1992) 89:7811-7815; Delgrave et al., Protein Engineering (1993) 6(3):327-331).

An isolated GAVE1 protein, or a portion or fragment thereof, can be used as an immunogen to generate antibodies that bind GAVE1 using standard techniques for polyclonal and monoclonal antibody preparation. The full-length GAVE1 protein can be used or, alternatively, the invention provides antigenic peptide fragments of GAVE1 for use as immunogens. The antigenic peptide of GAVE1 comprises at least 8 (preferably 10, 15, 20 or 30) amino acid residues of the amino acid sequence shown in SEQ ID NO: 2 and encompasses an epitope of GAVE1 such that an antibody raised against the peptide forms a specific immune complex with GAVE1. In one embodiment, an epitope may comprise an 8-mer comprising residues 250-252 of the third intracellular loop or residue 382 of the carboxyl terminus as set forth in SEQ ID NO: 2.

In a related aspect, epitopes encompassed by the antigenic peptide are regions of GAVE1 that are located on the surface of the protein, e.g., hydrophilic regions. A hydrophobicity analysis of the human GAVE1 protein sequence indicates that the regions between about amino acids 1 and about 46, between about amino acids 108 and about 121, between about amino acids 186 and about 201 and between about amino acids 278 and about 293 of SEQ ID NO: 2 are particularly hydrophilic and, therefore, are likely to encode surface residues useful for targeting antibody production.

A GAVE1 immunogen typically is used to prepare antibodies by immunizing a suitable subject, (e.g., rabbit, goat, mouse or other mammal) with the immunogen. An appropriate immunogenic preparation can contain, for example, recombinantly expressed GAVE1 protein or a chemically synthesized GAVE1 polypeptide. The preparation further can include an adjuvant, such as Freund's complete or incomplete adjuvant, or similar immunostimulatory agent. Immunization of a suitable subject with an immunogenic GAVE1 preparation induces a polyclonal anti-GAVE1 antibody response.

Accordingly, another aspect of the invention pertains to anti-GAVE1 antibodies. The term “antibody” as used herein refers to immunoglobulin molecules and immunologically active portions of immunoglobulin molecules, i.e., molecules that contain an antigen-binding site that specifically binds GAVE1. A molecule that specifically binds to GAVE1 is a molecule that binds GAVE1, but does not substantially bind other molecules in a sample, e.g., a biological sample, which naturally contains GAVE1. Examples of immunologically active portions of immunoglobulin molecules include F_{ab} and F_{(ab′)2} fragments which can be generated by treating the antibody with an enzyme such as pepsin. The invention provides polyclonal and monoclonal antibodies that bind GAVE1. The term “monoclonal antibody” or “monoclonal antibody composition”, as used herein, refers to a population of antibody molecules that contain only one species of an antigen binding site capable of immunoreacting with a particular epitope of GAVE1. A monoclonal antibody composition thus typically displays a single binding affinity for a particular GAVE1 protein with which it immunoreacts.

Polyclonal anti-GAVE1 antibodies can be prepared as described above by immunizing a suitable subject with a GAVE1 immunogen. The anti-GAVE1 antibody titer in the immunized subject can be monitored over time by standard techniques, such as with an enzyme linked immunosorbent assay (ELISA) using immobilized GAVE1.

If desired, the antibody molecules directed against GAVE1 can be isolated from the mammal (e.g., from the blood) and further purified by well-known techniques, such as protein A chromatography to obtain the IgG fraction. At an appropriate time after immunization, e.g., when the anti-GAVE1 antibody titers are highest, antibody-producing cells can be obtained from the subject and used to prepare monoclonal antibodies by standard techniques, such as the hybridoma technique originally described by Kohler et al., Nature (1975) 256:495-497, the human B cell hybridoma technique (Kozbor et al., Immunol Today (1983) 4:72), the EBV-hybridoma technique (Cole et al., Monoclonal Antibodies and Cancer Therapy, (1985), Alan R. Liss, Inc., pp. 77-96) or trioma techniques. The technology for producing hybridomas is well known (See generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with a GAVE1 immunogen as described above, and the culture supernatants of the resulting hybridoma cells are screened to identify a hybridoma producing a monoclonal antibody that binds GAVE1.

Any of the many well known protocols used for fusing lymphocytes and immortalized cell lines can be applied for the purpose of generating an anti-GAVE1 monoclonal antibody (See, e.g., Current Protocols in Immunology, supra; Galfre et al., Nature (1977) 266:55052. Kenneth, in Monoclonal Antibodies: A New Dimension In Biological Analyses, Plenum Publishing Corp., New York, N.Y. (1980); and Lerner, Yale J Biol Med (1981) 54:387-402). Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the instant invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/I-Ag4-1, P3×63-Ag8.653 or Sp2/O-Agl4 myeloma lines. Those myeloma lines are available from ATCC. Typically, HAT-sensitive mouse myeloma cells are fused to mouse splenocytes using polyethylene glycol (“PEG”). Hybridoma cells resulting from the fusion then are selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind GAVE1, e.g., using a standard ELISA assay.

Alternative to preparing monoclonal antibody-secreting hybridomas, a monoclonal anti-GAVE1 antibody can be identified and isolated by screening a recombinant combinatorial immunoglobulin library (e.g., an antibody phage display library) with GAVE1 to thereby isolate immunoglobulin library members that bind GAVE1. Kits for generating and screening phage display libraries are commercially available (e.g., the Pharmacia Recombinant Phage Antibody System, Catalog No. 27-9400-01; and the Stratagene SurfZAP®Phage Display Kit, Catalog No. 240612).

Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO90/02809; Fuchs et al., Bio/Technology (1991) 9:1370-1372; Hay et al., Hum Antibod Hybridomas (1992) 3:81-85; Huse et al., Science (1989) 246:1275-1281; and Griffiths et al., EMBO J (1993) 25 12:725-734.

Additionally, recombinant anti-GAVE1 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; Europe Patent Application 184,187; Europe Patent Application No. 171,496; Europe Patent Application No. 173,494; PCT Publication No. WO 86/01533; U.S. Pat. No. 4,816,567; Europe Patent Application No. 125,023; Better et al., Science (1988) 240:1041-1043; Liu et al., Proc Natl Acad Sci USA (1987) 84:3439-3443; Lin et al., J Immunol (1987) 139:3521-3526; Sun et al., Proc Natl Acad Sci USA (1987) 84:214-218; Nishimura et al., Canc Res (1987) 47:999-1005; Wood et al., Nature (1985) 314:446-449; Shaw et al., J Natl Cancer Inst (1988) 80:1553-1559; Morrison, Science (1985) 229:1202-1207; Oi et al., Bio/Techniques (1986) 4:214; U.S. Pat. No. 5,225,539; Jones et al., Nature (1986) 321:552-525; Verhoeyan et al., Science (1988) 239:1534; and Beidler et al., J Immunol (1988) 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of GAVE1. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such an epitope, e.g., an antibody that inhibits GAVE1 activity, is identified. The heavy chain and the light chain of the non-human antibody are cloned and used to create phage display F_{ab} fragments. For example, the heavy chain gene can be cloned into a plasmid vector so that the heavy chain can be secreted from bacteria. The light chain gene can be cloned into a phage coat protein gene so that the light chain can be expressed on the surface of phage. A repertoire (random collection) of human light chains fused to phage is used to infect the bacteria that express the non-human heavy chain. The resulting progeny phage display hybrid antibodies (human light chain/non-human heavy chain). The selected antigen is used in a panning screen to select phage which bind the selected antigen. Several rounds of selection may be required to identify such phage. Next, human light chain genes are isolated from the selected phage which bind the selected antigen. These selected human light chain genes are then used to guide the selection of human heavy chain genes as follows. The selected human light chain genes are inserted into vectors for expression by bacteria. Bacteria expressing the selected human light chains are infected with a repertoire of human heavy chains fused to phage. The resulting progeny phage display human antibodies (human light chain/human heavy chain).

Next, the selected antigen is used in a panning screen to select phage which bind the selected antigen. The phage selected in that step display a completely human antibody that recognizes the same epitope recognized by the original selected, non-human monoclonal antibody. The genes encoding both the heavy and light chains are isolated readily and can be manipulated further for production of human antibody. The technology is described by Jespers et al. (Bio/Technology (1994) 12:899-903).

An anti-GAVE1 antibody (e.g., monoclonal antibody) can be used to isolate GAVE1 by standard techniques, such as affinity chromatography or immunoprecipitation. An anti-GAVE1 antibody can facilitate the purification of natural GAVE1 from cells and of recombinantly produced GAVE1 expressed in host cells. Moreover, an anti-GAVE1 antibody can be used to detect GAVE1 protein (e.g., in a cellular lysate or cell supernatant) to evaluate the abundance and pattern of expression of the GAVE1 protein. Anti-GAVE1 antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials and radioactive materials. Examples of suitable enzymes include horseradish peroxidase, alkaline phosphatase, galactosidase or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, given fluorescent protein or phycoerythrin; an example of a luminescent material includes luminol; examples of bioluminescent materials include luciferase, luciferin, and aequorin, and examples of suitable radioactive materials include ¹²⁵I, ¹³¹I, ³⁵S or ³H.

### III. Recombinant Expression Vectors and Host Cells

Another aspect of the invention pertains to vectors, preferably expression vectors, containing a nucleic acid encoding GAVE1 (or a portion thereof). As used herein, the term “vector” refers to a nucleic acid molecule capable of transporting another nucleic acid to which it has been linked. One type of vector is a “plasmid”, which refers to a circular double-stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), that serve equivalent functions.

The recombinant expression vectors of the invention comprise nucleic acid of the invention in a form suitable for expression of the nucleic acid in a host cell. That means that the recombinant expression vectors include one or more regulatory sequences, selected on the basis of host cells to be used for expression, which is operably linked to the nucleic acid to be expressed. Within a recombinant expression vector, “operably linked” is intended to mean that the nucleotide sequence of interest is linked to the regulatory sequence(s) in a manner which allows for expression of the nucleotide sequence (e.g., in an in vivo transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology in Methods in Enzymology Vol. 185, Academic Press, San Diego, Calif. (1990). Regulatory sequences include those that direct constitutive expression of the nucleotide sequence in many types of host cells (e.g., tissue specific regulatory sequences). It will be appreciated by those skilled in the art that the design of the expression vector can depend on such factors as the choice of host cell to be transformed, the level of expression of protein desired etc. The expression vectors of the invention can be introduced into host cells to thereby produce proteins or peptides, encoded by nucleic acids as described herein (e.g., GAVE1 proteins, mutant forms of GAVE1, fusion proteins etc.).

The recombinant expression vectors of the invention can be designed for expression of GAVE1 in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith et al., Gene (1988) 67:31-40), pMAL (New England Biolabs, Beverly, Mass.) and pRITS (Pharmacia, Piscataway, N.J.) which fuse glutathione 5-transferase (GST), maltose E binding protein or protein A, respectively, to the target recombinant protein.

Examples of suitable inducible non-fusion E. coli expression vectors include pTrc (Amann et al., Gene (1988) 69:301-315) and pET 11d (Studier et al., Gene Expression Technology in Methods in Enzymology, Academic Press, San Diego, Calif. (1990) 185:60-89). Target gene expression from the pTrc vector relies on host RNA polymerase transcription from a hybrid trp-lac fusion promoter. Target gene expression from the pET 11d vector relies on transcription from a T7 gnl-lac fusion promoter mediated by a coexpressed viral RNA polymerase (T7 gnl). That viral polymerase is supplied by host strains BL21 (DE3) or HMS 174 (DE3) from a resident λ prophage harboring a T7 gn 1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology in Methods in Enzymology, Academic Press, San Diego, Calif. (1990) 185:119-128). Another strategy is to alter the nucleic acid sequence of the nucleic acid to be inserted into an expression vector so that the individual codons for each amino acid are those preferentially utilized in E. coli (Wada et al., Nucleic Acids Res (1992) 20:2111-2118). Such alteration of nucleic acid sequences of the invention can be carried out by standard DNA synthesis techniques.

In another embodiment, the GAVE1 expression vector is a yeast expression vector. Examples of vectors for expression in yeast S. cerevisiae include pYepSecl (Baldari et al., EMBO J (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.) and pPicZ (Invitrogen Corp, San Diego, Calif.).

Alternatively, GAVE1 can be expressed in insect cells using baculovirus expression vectors. Baculovirus vectors available for expression of proteins in cultured insect cells (e.g., Sf9 cells) include the pAc series (Smith et al., Mol Cell Biol (1983) 3:2156-2165) and the pVL series (Lucklow et al., Virology (1989) 170:31-39).

In yet another embodiment, a nucleic acid of the invention is expressed in mammalian cells using a mammalian expression vector. Examples of mammalian expression vectors include pCDM8 (Seed, Nature (1987) 329:840) and pMT2PC (Kaufman et al., EMBO J (1987) 6:187-195). When used in mammalian cells, the control functions of the expression vector often are provided by viral regulatory elements. For example, commonly used promoters are derived from polyoma, Adenovirus 2, cytomegalovirus and Simian Virus 40. For other suitable expression systems for both prokaryotic and eukaryotic cells, see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., Genes Dev (1987) 1:268-277), lymphoid-specific promoters (Calame et al., Adv Immunol (1988) 43:235-275), in particular promoters of T cell receptors (Winoto et al., EMBO J (1989) 8:729-733) and immunoglobulins (Banerji et al., Cell (1983) 33:729-740; Queen et al., Cell (1983) 33:741-748), neuron-specific promoters (e.g., the neurofilament promoter; Byrne et al., Proc Natl Acad Sci USA (1989) 86:5473-5477), pancreas-specific promoters (Edlund et al., Science (1985) 230:912-916) and mammary gland-specific promoters (e.g., milk whey promoter; U.S. Pat. No. 4,873,316 and Europe Patent Application No. 264,166). Developmentally-regulated promoters also are encompassed, for example the murine hox promoters (Kessel et al., Science (1990) 249:374-379) and the α-fetoprotein promoter (Campes et al., Genes Dev (1989) 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule that is antisense to GAVE1 mRNA. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub et al. (Reviews—Trends in Genetics, Vol. 1(1)1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but still are included within the scope of the term as used herein.

A host cell can be any prokaryotic or eukaryotic cell. For example, GAVE1 protein can be expressed in bacterial cells such as E. coli, insect cells, yeast or mammalian cells (such as Chinese hamster ovary cells (CHO) or COS cells). Other suitable host cells are known to those skilled in the art. Vector DNA can be introduced into prokaryotic or eukaryotic cells via conventional transformation or transfection techniques. As used herein, the terms “transformatio” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection or electroporation.

For stable transfection of mammalian cells, it is known that, depending on the expression vector and transfection technique used, only a small fraction of cells may integrate the foreign DNA into the genome. To identify and to select those integrants, a gene that encodes a selectable marker (e.g., for resistance to antibiotics) generally is introduced into the host cells along with the gene of interest. Preferred selectable markers include those that confer resistance to drugs, such as G418, hygromycin and methotrexate. Nucleic acid encoding a selectable marker can be introduced into a host cell on the same vector as that encoding GAVE1 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GAVE1 protein. Accordingly, the invention further provides methods for producing GAVE1 protein using the host cells of the invention. In one embodiment, the method comprises culturing the host cell of invention (into which a recombinant expression vector encoding GAVE1 has been introduced) in a suitable medium such that GAVE1 protein is produced. In another embodiment, the method further comprises isolating GAVE1 from the medium or the host cell.

The host cells of the invention also can be used to produce nonhuman transgenic animals. For example, in one embodiment, a host cell of the invention is a fertilized oocyte or an embryonic stem cell into which GAVE1-coding sequences have been introduced. Such host cells then can be used to create non-human transgenic animals in which exogenous GAVE1 sequences have been introduced into the genome or homologous recombinant animals in which endogenous GAVE1 sequences have been altered. Such animals are useful for studying the function and/or activity of GAVE1 and for identifying and/or evaluating modulators of GAVE1 activity. As used herein, a “transgenic animal” preferably is a mammal, more preferably, a rodent such as a rat or mouse, in which one or more of the cells of the animal includes a transgene. Other examples of transgenic animals include non-human primates, sheep, dogs, cows, goats, chickens, amphibians etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” preferably is a mammal, more preferably, a mouse, in which an endogenous GAVE1 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GAVE1 -encoding nucleic acid into the male pronuclei of a fertilized oocyte, e.g., by microinjection, retroviral infection and allowing the oocyte to develop in a pseudopregnant female foster animal. The GAVE1 cDNA sequence e.g., that of SEQ ID NO: 1, can be introduced as a transgene into the genome of a non-human animal. Alternatively, a nonhuman homologue of the human GAVE1 gene, such as a mouse GAVE1 gene, can be isolated based on hybridization to the human GAVE1 cDNA and used as a transgene. Intronic sequences and polyadenylation signals also can be included in the transgene to increase the efficiency of expression of the transgene. A tissue-specific regulatory sequence(s) can be operably linked to the GAVE1 transgene to direct expression of GAVE1 protein to particular cells. Methods for generating transgenic animals via embryo manipulation and microinjection, particularly animals such as mice, are conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y.,1986). Similar methods are used for production of other transgenic animals. A transgenic founder animal then can be used to breed additional animals carrying the transgene. Moreover, transgenic animals carrying a transgene encoding GAVE1 further can be bred to other transgenic animals carrying other transgenes.

To create a homologous recombinant animal, a vector is prepared which contains at least a portion of a GAVE1 gene (e.g., a human or a non-human homolog of the GAVE1 gene, e.g., a murine GAVE1 gene) into which a deletion, addition or substitution has been introduced to thereby alter, e.g., functionally disrupt, the GAVE1 gene. In a preferred embodiment, the vector is designed such that, on homologous recombination, the endogenous GAVE1 gene is functionally disrupted (i.e., no longer encodes a functional protein; also referred to as a “knock out” animal). Alternatively, the vector can be designed such that, on homologous recombination, the endogenous GAVE1 gene is mutated or otherwise altered but still encodes functional protein (e.g., the upstream regulatory region can be altered to thereby alter the expression of the endogenous GAVE1 protein). In the homologous recombination vector, the altered portion of the GAVE1 gene is flanked at the 5′ and 3′ ends by additional nucleic acid of the GAVE1 gene to allow for homologous recombination to occur between the exogenous GAVE1 gene carried by the vector and an endogenous GAVE1 gene in an embryonic stem cell. The additional flanking GAVE1 nucleic acid is of sufficient length for successful homologous recombination with the endogenous gene. Typically, several kilobases of flanking DNA (both at the 5′ and 3′ ends) are included in the vector (See, e.g., Thomas et al., Cell (1987) 51:503 for a description of homologous recombination vectors). The vector is introduced into an embryonic stem cell line (e.g., by electroporation) and cells in which the introduced GAVE1 gene has homologously recombined with the endogenous GAVE1 gene are selected (See, e.g., Li et al., Cell (1992) 69:915). The selected cells then are injected into a blastocyst of an animal (e.g., a mouse) to form aggregation chimeras (See, e.g., Bradley in Teratocarcinomas and Embryonic Stem Cells: A Practical Approach, Robertson, ed., IRL, Oxford, (1987) pp. 113-152). A chimeric embryo then can be implanted into a suitable pseudopregnant female foster animal and the embryo brought to term. Progeny harboring the homologously recombined DNA in germ cells can be used to breed animals in which all cells of the animal contain the homologously recombined DNA by germline transmission of the transgene. Methods for constructing homologous recombination vectors and homologous recombinant animals are described further in Bradley, Current Opinion in Bio/Technology (1991) 2:823-829 and in PCT Publication Nos. WO 90/11354, WO 91/01140, WO 92/0968 and WO 93/04169.

In another embodiment, transgenic non-human animals can be produced which contain selected systems that allow for regulated expression of the transgene. One example of such a system is the cre/loxP recombinase system of bacteriophage P1. For a description of the cre/loxP recombinase system, see, e.g., Lakso et al., Proc Natl Acad Sci USA (1992) 89:6232-6236. Another example of a recombinase system is the FLP recombinase system of S. cerevisiae (O'Gorrnan et al., Science (1991) 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein also can be produced according to the methods described in Wilmut et al., Nature (1997) 385:810-813 and PCT Publication Nos. WO 97/07668 and WO 97/07669. In brief, a cell, e.g., a somatic cell, from the transgenic animal can be isolated and induced to exit the growth cycle and enter GO phase. The quiescent cell then can be fused, e.g., through the use of electrical pulses, to an enucleated oocyte from an animal of the same species from which the quiescent cell is isolated. The reconstructed oocyte then is cultured such that it develops to morula or blastocyst and then transferred to a pseudopregnant female foster animal. The offspring borne of that female foster animal will be a clone of the animal from which the cell, e.g., the somatic cell, is isolated.

### IV. Pharmaceutical Compositions

The GAVE1 nucleic acid molecules, GAVE1 proteins and anti-GAVE1 antibodies (also referred to herein as “active compounds”) of the invention can be incorporated into pharmaceutical compositions suitable for administration. Such compositions typically comprise the nucleic acid molecule, protein or antibody and a pharmaceutically acceptable carrier. As used herein, the language, “pharmaceutically acceptable carrier,” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds also can be incorporated into the compositions.

A pharmaceutical composition of the invention is formulated to be compatible with the intended route of administration. Examples of routes of administration include parenteral, e.g., intravenous, intradermal, subcutaneous, oral (e.g., inhalation), transdermal (topical), transmucosal and rectal administration. Solutions or suspensions used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose. Acidity (pH) can be adjusted with acids or bases, such as HCl or NaOH. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL® (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyetheylene glycol and the like) and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a GAVE1 protein or anti-GAVE1 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above.

In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches or capsules. Oral compositions also can be prepared using a fluid carrier for use as a mouthwash, wherein the compound in the fluid carrier is applied orally, swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels or creams, as generally known in the art.

The compounds also can be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparing such formulations will be apparent to those skilled in the art. The materials also can be obtained commercially from Alza Corporation and Nova Pharmaceuticals, Inc. Liposomal suspensions (including liposomes targeted to infected cells with monoclonal antibodies to viral antigens) also can be used as pharmaceutically acceptable carriers. Those can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited to unitary dosages, each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of the therapy is monitored easily by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals. The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al., Proc Natl Acad Sci USA (1994) 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack or dispenser, together with instructions for administration.

### V. Uses and Methods of the Invention

The nucleic acid molecules, proteins, protein homologues and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials and pharmacogenomics); and d) methods of treatment (e.g., therapeutic and prophylactic). A GAVE1 protein interacts with other cellular proteins and can thus be used for (i) regulation of cellular proliferation; (ii) regulation of cellular differentiation; and (iii) regulation of cell survival. The isolated nucleic acid molecules of the invention can be used to express GAVE1 protein (e.g., via a recombinant expression vector in a host cell in gene therapy applications), to detect GAVE1 mRNA (e.g., in a biological sample) or a genetic lesion in a GAVE1 gene, and to modulate GAVE1 activity. In addition, the GAVE1 proteins can be used to screen drugs or compounds which modulate the GAVE1 activity or expression as well as to treat disorders characterized by insufficient or excessive production of GAVE1 protein or production of GAVE1 protein forms which have decreased or aberrant activity compared to GAVE1 wild-type protein. In addition, the anti-GAVE1 antibodies of the invention can be used to detect and to isolate GAVE1 proteins and to modulate GAVE1 activity. The invention further pertains to novel agents identified by the above-described screening assays and uses thereof for treatments as described herein.

### A. Screening Assays

The invention provides a method (also referred to herein as a “screening assay”) for identifying modulators, i.e., candidate or test compounds or agents (e.g., peptides, peptidomimetics, small molecules or other drugs) which bind to GAVE1 proteins or have a stimulatory or inhibitory effect on, for example, GAVE1 expression or GAVE1 activity.

In one embodiment, the invention provides assays for screening candidate or test compounds which bind to or modulate the activity of the membrane-bound form of a GAVE1 protein or polypeptide or biologically active portion thereof. The test compounds of the instant invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des (1997) 12:145).

Examples of methods for the synthesis of molecular libraries can be found in the art, for example in: DeWitt et al., Proc Natl Acad Sci USA (1993) 90:6909; Erb et al., Proc Natl Acad Sci USA (1994) 91:11422; Zuckermann et al., J Med Chem (1994) 37:2678; Cho et al., Science (1993) 261:1303; Carrell et al., Angew Chem Int Ed Engl (1994) 33:2059; Carell et al., Angew Chem Int Ed Engl (1994) 33:2061; and Gallop et al., J Med Chem (1994) 37:1233.

Libraries of compounds may be presented in solution (e.g., Houghten, Bio/Techniques (1992) 13:412-421), or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al., Proc Natl Acad Sci USA (1992) 89:1865-1869) or phage (Scott et al., Science (1990) 249:386-390; Devlin, Science (1990) 249:404-406; Cwirla et al., Proc Natl Acad Sci USA (1990) 87:6378-6382; and Felici, J Mol Biol (1991) 222:301-310).

Because a GAVE1 ligand is S1 P, S1P can be investigated to determine the particular portion thereof that engages GAVE1, practicing known methods. That particular region can be synthesized practicing known biosynthetic methods, combining carbohydrate synthesis and enzymatic reactions, for example. That portion of S1P is equivalent to an “epitope.” The GAVE1 epitope can be modified using other monomers or non-carbohydrate moities to yield modified epitopes with enhanced properties, such as serum half-life, binding constant with GAVE1 and so on. The suitability of any one epitope variant can be determined practicing the binding and screening assays taught herein.

In one embodiment, an assay is a cell-based assay in which a cell which expresses a membrane-bound form of GAVE1 protein, or a biologically active portion thereof, on the cell surface is contacted with a test compound and the ability of the test compound to bind to a GAVE1 protein determined. The cell, for example, can be a yeast cell or a cell of mammalian origin. Determining the ability of the test compound to bind to the GAVE1 protein can be accomplished, for example, by coupling the test compound with a radioisotope or enzymatic label such that binding of the test compound to the GAVE1 protein or biologically active portion thereof can be determined by detecting the labeled compound in a complex. For example, test compounds can be labeled with ¹²⁵I, ³⁵S, ¹⁴C or ³H, either directly or indirectly, and the radioisotope detected by direct counting of radioemmission or by scintillation counting. Alternatively, test compounds can be labeled enzymatically with, for example, horseradish peroxidase, alkaline phosphatase or luciferase, and the enzymatic label detected by determination of conversion of an appropriate substrate to product. In a preferred embodiment, the assay comprises contacting a cell which expresses a membrane-bound form of GAVE1 protein, or a biologically active portion thereof, on the cell surface with a known compound which binds GAVE1 to form an assay mixture, contacting the assay mixture with a test compound, and determining the ability of the test compound to interact with a GAVE1 protein, wherein determining the ability of the test compound to interact with a GAVE1 protein comprises determining the ability of the test compound to preferentially bind to GAVE1 or a biologically active portion thereof as compared to the known compound.

In another embodiment, an assay is a cell-based assay comprising contacting a cell expressing a membrane-bound form of GAVE1 protein, or a biologically active portion thereof, on the cell surface with a test compound and determining the ability of the test compound to modulate (e.g., stimulate or inhibit) the activity of the GAVE1 protein or biologically active portion thereof. Determining the ability of the test compound to modulate the activity of GAVE1 or a biologically active portion thereof can be accomplished, for example, by determining the ability of the GAVE1 protein to bind to or interact with a GAVE1 target molecule. As used herein, a “target molecule” is a molecule with which a GAVE1 protein binds or interacts in nature, for example, a molecule on the surface of a cell which expresses a GAVE1 protein, a molecule on the surface of a second cell, a molecule in the extracellular milieu, a molecule associated with the internal surface of a cell membrane or a cytoplasmic molecule. A GAVE1 target molecule can be a non-GAVE1 molecule or a GAVE1 protein or polypeptide of the instant invention. In one embodiment, a GAVE1 target molecule is a component of a signal transduction pathway that facilitates transduction of an extracellular signal (e.g., a signal generated by binding of a compound to a membrane-bound GAVE1 molecule) through the cell membrane and into the cell. The target, for example, can be a second intercellular protein that has catalytic activity or a protein that facilitates the association of downstream signaling molecules with GAVE1.

In another embodiment, GAVE1 is made to signal constitutively using known techniques, see, for example WO 00/22131 and WO 00/22129, expressed in a target cell as taught herein, and then the cell is exposed to various candidate modulators to determine if signaling activity, the monitoring of which is described herein, is enhanced, revealing a candidate agonist, or diminished, revealing a candidate antagonist, or if activity is reduced below baseline levels, a candidate inverse agonist.

Determining the ability of the GAVE1 protein to bind to or to interact with a GAVE1 target molecule can be accomplished by one of the methods described above for determining direct binding. In a preferred embodiment, determining the ability of the GAVE1 protein to bind to or to interact with a GAVE1 target molecule can be accomplished by determining the activity of the target molecule. For example, the activity of the target molecule can be determined by detecting induction of a cellular second messenger of the target (e.g., intracellular Ca⁺², diacylglycerol, IP3 etc.), detecting catalytic/enzymatic activity of the target on an appropriate substrate, detecting the induction of a reporter gene (e.g., a GAVE1-responsive regulatory element operably linked to a nucleic acid encoding a detectable marker, e.g. luciferase), or detecting a cellular response, for example, cellular differentiation or cell proliferation.

In yet another embodiment, an assay of the instant invention is a cell-free assay comprising contacting a GAVE1 protein or biologically active portion thereof with a test compound and determining the ability of the test compound to bind to the GAVE1 protein or biologically active portion thereof. Binding of the test compound to the GAVE1 protein can be determined either directly or indirectly as described above. In a preferred embodiment, the assay includes contacting the GAVE1 protein or biologically active portion thereof with a known compound which binds GAVE1 to form an assay mixture, contacting the assay mixture with a test compound, and determining the ability of the test compound to interact with a GAVE1 protein, wherein determining the ability of the test compound to interact with a GAVE1 protein comprises determining the ability of the test compound to preferentially bind to GAVE1 or a biologically active portion thereof, as compared to the known compound.

In another embodiment, an assay is a cell-free assay comprising contacting GAVE1 protein or biologically active portion thereof with a test compound and determining the ability of the test compound to modulate (e.g., stimulate or inhibit) the activity of the GAVE1 protein or a biologically active portion thereof. Determining the ability of the test compound to modulate the activity of GAVE1 can be accomplished, for example, by determining the ability of the GAVE1 protein to bind to a GAVE1 target molecule by one of the methods described above for determining direct binding. In an alternative embodiment, determining the ability of the test compound to modulate the activity of GAVE1 can be accomplished by determining the ability of the GAVE1 protein to further modulate a GAVE1 target molecule. For example, the catalytic/enzymatic activity of the target molecule on an appropriate substrate can be determined as described previously.

In yet another embodiment, the cell-free assay comprises contacting the GAVE1 protein or biologically active portion thereof with a known compound which binds GAVE1 to form an assay mixture, contacting the assay mixture with a test compound, and determining the ability of the test compound to interact with a GAVE1 protein, wherein determining the ability of the test compound to interact with a GAVE1 protein comprises determining the ability of the GAVE1 protein to preferentially bind to or modulate the activity of a GAVE1 target molecule.

The cell-free assays of the instant invention are amenable to use of both the soluble form and the membrane-bound form of GAVE1. In the case of cell-free assays comprising the membrane-bound form of GAVE1, it may be desirable to utilize a solubilizing agent such that the membrane-bound form of GAVE1 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit®, isotridecylpoly(ethyleneglycol-ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO) or N-dodecyl=N,N-dimethyl-3-ammonio-1-propane sulfonate.

In more than one embodiment of the above assay methods of the instant invention, it may be desirable to immobilize either GAVE1 or a target molecule to facilitate separation of complexed from uncomplexed forms of one or both of the proteins, as well as to accommodate automation of the assay. Binding of a test compound to GAVE1, or interaction of GAVE1 with a target molecule in the presence and absence of a candidate compound, can be accomplished in any vessel suitable for containing the reactants. Examples of such vessels include microtitre plates, test tubes and micro-centrifuge tubes. In one embodiment, a fusion protein can be provided which adds a domain that allows one or both of the proteins to be bound to a matrix. For example, glutathione-S-transferase/GAVE1 fusion proteins or glutathione-S-transferase/target fusion proteins can be adsorbed onto glutathione Sepharose beads (Sigma Chemical, St. Louis, Mo.) or glutathione derivatized microtitre plates, which then are combined with the test compound or the test compound and either the non-adsorbed target protein or GAVE1 protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix and the level of GAVE1 binding or activity determined using standard techniques.

Other techniques for immobilizing proteins on matrices also can be used in the screening assays of the invention. For example, either GAVE1 or its target molecule can be immobilized utilizing conjugation of biotin and streptavidin. Biotinylated GAVE1 or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemicals). Alternatively, antibodies reactive with GAVE1 or target molecules but which do not interfere with binding of the GAVE1 protein to a target molecule can be derivatized to the wells of the plate, and unbound target or GAVE1 trapped in the wells by antibody conjugation. Methods for detecting such complexes, in addition to those described above for the GST-immobilized complexes, include immunodetection of complexes using antibodies reactive with the GAVE1 or target molecule, as well as enzyme-linked assays which rely on detecting an enzymatic activity associated with the GAVE1 or target molecule.

In another embodiment, modulators of GAVE1 expression are identified in a method in which a cell is contacted with a candidate compound and the expression of GAVE1 mRNA or protein in the cell is determined. The level of expression of GAVE1 mRNA or protein in the presence of the candidate compound is compared to the level of expression of GAVE1 mRNA or protein in the absence of the candidate compound. The candidate compound then can be identified as a modulator of GAVE1 expression based on that comparison. For example, when expression of GAVE1 mRNA or protein is greater (statistically significantly greater) in the presence of the candidate compound than in its absence, the candidate compound is identified as a stimulator of GAVE1 mRNA or protein expression. Alternatively, when expression of GAVE1 mRNA or protein is less (statistically significantly less) in the presence of the candidate compound than in its absence, the candidate compound is identified as an inhibitor of GAVE1 mRNA or protein expression. The level of GAVE1 mRNA or protein expression in the cells can be determined by methods described herein for detecting GAVE1 mRNA or protein.

In yet another aspect of the invention, the GAVE1 proteins can be used as “bait proteins” in a two-hybrid assay or three hybrid assay (see, e.g., U.S. Pat. No. 5,283,317; Zervos et al., Cell (1993) 72:223-232; Madura et al., J Biol Chem (1993) 268:12046-12054; Bartel et al., Bio/Techniques (1993) 14:920-924; Iwabuchi et al., OncoGene (1993) 8:1693-1696; and PCT Publication No. WO 94/10300), to identify other proteins, which bind to or interact with GAVE1 (“GAVE1-binding proteins” or “GAVE1-bp”) and modulate GAVE1 activity. Such GAVE1-binding proteins are also likely to be involved in the propagation of signals by the GAVE1 proteins as, for example, upstream or downstream elements of the GAVE1 pathway.

The invention further pertains to novel agents identified by the above-described screening assays and uses thereof for treatments as described herein.

### B. Detection Assays

Portions or fragments of the cDNA sequences identified herein (and the corresponding complete gene sequences) can be used in numerous ways as polynucleotide reagents. For example, the sequences can be used to: (i) map respective genes on a chromosome and, thus, locate gene regions associated with genetic disease; (ii) identify an individual from a minute biological sample (tissue typing); and (iii) aid in forensic identification of a biological sample. Those applications are described in the subsections below.

### I. Chromosome Mapping

Once the sequence (or a portion of the sequence) of a gene has been isolated, the sequence can be used to map the location of the gene on a chromosome. Accordingly, GAVE1 nucleic acid molecules described herein or fragments thereof; can be used to map the location of GAVE1 genes on a chromosome. The mapping of the GAVE1 sequences to chromosomes is an important first step in correlating the sequences with genes associated with disease.

Briefly, GAVE1 genes can be mapped to chromosomes by preparing PCR primers (preferably 15-25 bp in length) from the GAVE1 sequences. Computer analysis of GAVE1 sequences can be used to select primers that do not span more than one exon in the genomic DNA, which could complicate the amplification process. The primers then can be used for PCR screening of somatic cell hybrids containing individual human chromosomes. Only those hybrids containing the human gene corresponding to the GAVE1 sequences will yield an amplified fragment.

Somatic cell hybrids are prepared by fusing somatic cells from different mammals (e.g., human and mouse cells). As hybrids of human and mouse cells grow and divide, the cells gradually lose human chromosomes in random order, but retain the mouse chromosomes. By using media in which mouse cells cannot grow (due to lack of a particular selecting enzyme), but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al., Science (1983) 220:919-924). Somatic cell hybrids containing only fragments of human chromosomes also can be produced by using human chromosomes with translocations and deletions.

PCR mapping of somatic cell hybrids is a rapid procedure for assigning a particular sequence to a particular chromosome. Using the GAVE1 sequences to design oligonucleotide primers, sublocalization can be achieved with panels of fragments from specific chromosomes. Other mapping strategies which can similarly be used to map a GAVE1 sequence to its chromosome include in situ hybridization (described in Fan et al., Proc Natl Acad Sci USA (1990) 87:6223-27), pre-screening with labeled flow-sorted chromosomes and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical, e.g., colcemid, which disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of the technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to non-coding regions of the genes also can be used for mapping purposes. Some coding sequences are conserved within gene families, thus increasing the chance of cross hybridization during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, then can be identified through linkage analysis (co-inheritance of physically adjacent genes), described in, e.g., Egeland et al., Nature (1987) 325:783-787.

Moreover, differences in the DNA sequences between individuals affected and unaffected with a disease associated with the GAVE1 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### 2. Tissue Typing

The GAVE1 sequences of the instant invention also can be used to identify individuals from minute biological samples. The United States military, for example, is considering the use of restriction fragment length polymorphism (RFLP) for identification of personnel. In that technique, genomic DNA of an individual is digested with one or more restriction enzymes, and probed on a Southern blot to yield unique bands for identification. The method does not suffer from the current limitations of “Dog Tags” which can be lost, switched or stolen, making positive identification difficult. The sequences of the instant invention are useful as additional DNA markers for RFLP (described in U.S. Pat. No. 5,272,057).

Furthermore, the sequences of the instant invention can be used to provide an alternative technique that determines the actual base-by-base DNA sequence of selected portions of a genome. Thus, the GAVE1 sequences described herein can be used to prepare two PCR primers from the 5′ and 3 ends of the sequences. The primers then can be used to amplify genomic DNA and subsequently provide the sequence thereof.

Panels of corresponding DNA sequences from individuals, prepared in that manner, can provide unique individual identifications, as each individual will have a unique set of such DNA sequences due to allelic differences. The sequences of the instant invention can be used to obtain such identification sequences from individuals and from tissue. The GAVE1 sequences of the invention uniquely represent portions of the human genome. Allelic variation occurs to some degree in the coding regions of the sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1 can provide positive individual identification with a panel of perhaps 10 to 1,000 primers which each yield a noncoding amplified sequence of 100 bases. If predicted coding sequences, such as those in SEQ ID NO: 1 are used, a more appropriate number of primers for positive individual identification would be 500-2,000.

If a panel of reagents from GAVE1 sequences described herein is used to generate a unique identification database for an individual, those same reagents can later be used to identify tissue from that individual. Using the unique identification database, positive identification of the individual, living or dead, can be made from extremely small tissue samples.

### 3. Use of Partial GAVE1 Sequences in Forensic Biology

DNA-based identification techniques also can be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva or semen, found at a crime scene. The amplified sequence then can be compared to a standard, thereby allowing identification of the origin of the biological sample.

The sequences of the instant invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1 are particularly appropriate for that use as greater numbers of polymorphisms occur in the noncoding regions, making it easier to differentiate individuals using that technique. Examples of polynucleotide reagents include the GAVE1 sequences or portions thereof, e.g., fragments derived from the noncoding regions of SEQ ID NO: 1 having a length of at least 20 or 30 bases.

The GAVE1 sequences described herein can be used further to provide polynucleotide reagents, e.g., labeled or labelable probes which can be used in, for example, an in situ hybridization technique, to identify a specific tissue, e.g., brain tissue. That can be very useful in cases where a forensic pathologist is presented with a tissue of unknown origin. Panels of such GAVE1 probes can be used to identify tissue by species and/or by organ type.

In a similar fashion, the reagents, e.g., GAVE1 primers or probes can be used to screen tissue culture for contamination (i.e., screen for the presence of a mixture of different types of cells in a culture).

### B. Predictive Medicine

The instant invention also pertains to the field of predictive medicine in which diagnostic assays, prognostic assays, pharmacogenomics and monitoring clinical trails are used for prognostic (predictive) purposes to treat thereby an individual prophylactically. Accordingly, one aspect of the instant invention relates to diagnostic assays for determining GAVE1 protein and/or nucleic acid expression as well as GAVE1 activity, in the context of a biological sample (e.g., blood, urine, feces, serum, cells, tissue) to determine thereby whether an individual is afflicted with a disease or disorder, or is at risk of developing a disorder, associated with aberrant GAVE1 expression or activity. The invention also provides for prognostic (or predictive) assays for determining whether an individual is at risk of developing a disorder associated with GAVE1 protein, nucleic acid expression or activity. For example, mutations in a GAVE1 gene can be assayed in a biological sample. Such assays can be used for prognostic or predictive purpose to thereby prophylactically treat an individual prior to the onset of a disorder characterized by or associated with GAVE1 protein, nucleic acid expression or activity.

Another aspect of the invention provides methods for determining GAVE1 protein, nucleic acid expression or GAVE1 activity in an individual to select thereby appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of GAVE1 in clinical trials.

Those and other agents are described in further detail in the following sections.

### 1. Diagnostic Assays

An exemplary method for detecting the presence or absence of GAVE1 in a biological sample involves obtaining a biological sample from a test subject and contacting the biological sample with a compound or an agent capable of detecting GAVE1 protein or nucleic acid (e.g., mRNA, genomic DNA) that encodes GAVE1 protein such that the presence of GAVE1 is detected in the biological sample. A preferred agent for detecting GAVE1 mRNA or genomic DNA is a labeled nucleic acid probe capable of hybridizing to GAVE1 mRNA or genomic DNA. The nucleic acid probe can be, for example, a full-length GAVE1 nucleic acid, such as the nucleic acid of SEQ ID NO: 1 or a portion thereof, such as an oligonucleotide of at least 15, 30, 50, 100, 250 or 500 nucleotides in length and sufficient to specifically hybridize under stringent conditions to GAVE1 mRNA or genomic DNA. Other suitable probes for use in the diagnostic assays of the invention are described herein.

A preferred agent for detecting GAVE1 protein is an antibody capable of binding to GAVE1 protein, preferably an antibody with a detectable label. Antibodies can be polyclonal, or more preferably, monoclonal. An intact antibody, or a fragment thereof (e.g., F_{ab} or F_{(ab′)2}) can be used. The term, “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect GAVE1 mRNA, protein or genomic DNA in a biological sample in vivo as well as in vitro. For example, in vivo techniques for detection of GAVE1 mRNA include Northern hybridization and in situ hybridization. In vivo techniques for detection of GAVE1 protein include enzyme linked immunosorbent assay (ELISAs), Western blot, immunoprecipitation and immunofluorescence. In vitro techniques for detection of GAVE1 genomic DNA include Southern hybridizations. Furthermore, in vivo techniques for detection of GAVE1 protein include introducing into a subject a labeled anti-GAVE1 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting GAVE1 protein, mRNA or genomic DNA, such that the presence of GAVE1 protein, mRNA or genomic DNA is detected in the biological sample, and comparing the presence of GAVE1 protein, mRNA or genomic DNA in the control sample with the presence of GAVE1 protein, mRNA or genomic DNA in the test sample.

The invention also encompasses kits for detecting the presence of GAVE1 in a biological sample (a test sample). Such kits can be used to determine if a subject is suffering from or is at increased risk of developing a disorder associated with aberrant expression of GAVE1 (e.g., an immunological disorder). For example, the kit can comprise a labeled compound or agent capable of detecting GAVE1 protein or mRNA in a biological sample and means for determining the amount of GAVE1 in the sample (e.g., an anti-GAVE1 antibody or an oligonucleotide probe which binds to DNA encoding GAVE1, e.g., SEQ ID NO: 1). Kits also can include instructions for observing that the tested subject is suffering from or is at risk of developing a disorder associated with aberrant expression of GAVE1, if the amount of GAVE1 protein or mRNA is above or below a normal level.

For antibody-based kits, the kit can comprise, for example: (1) a first antibody (e.g., attached to a solid support) which binds to GAVE1 protein; and, optionally, (2) a second, different antibody which binds to GAVE1 protein or the first antibody and is conjugated to a detectable agent.

For oligonucleotide-based kits, the kit can comprise, for example: (1) an oligonucleotide, e.g., a detectably labeled oligonucleotide, which hybridizes to a GAVE1 nucleic acid sequence or (2) a pair of primers useful for amplifying a GAVE1 nucleic acid molecule.

The kit also can comprise, e.g., a buffering agent, a preservative or a protein stabilizing agent. The kit also can comprise components necessary for detecting the detectable agent (e.g., an enzyme or a substrate). The kit also can contain a control sample or a series of control samples that can be assayed and compared to the test sample contained. Each component of the kit usually is enclosed within an individual container and all of the various containers are within a single package along with instructions for observing whether the tested subject is suffering from or is at risk of developing a disorder associated with aberrant expression of GAVE1.

### 2. Prognostic Assays

The methods described herein furthermore can be utilized as diagnostic or prognostic assays to identify subjects having or are at risk of developing a disease or disorder associated with aberrant GAVE1 expression or activity. For example, the assays described herein, such as the preceding diagnostic assays or the following assays, can be utilized to identify a subject having or is at risk of developing a disorder associated with GAVE1 protein, nucleic acid expression or activity, e.g., recent inflammation associated with cardiac ischemia. Alternatively, the prognostic assays can be utilized to identify a subject having or is at risk for developing such a disease or disorder. Thus, the instant invention provides a method in which a test sample is obtained from a subject and GAVE1 protein or nucleic acid (e.g., mRNA, genomic DNA) is detected, wherein the presence of GAVE1 protein or nucleic acid is diagnostic for a subject having or is at risk of developing a disease or disorder associated with aberrant GAVE1 expression or activity. As used herein, a “test sample” refers to a biological sample obtained from a subject of interest. For example, a test sample can be a biological fluid (e.g., serum), cell sample or tissue. Furthermore, the prognostic assays described herein can be used to determine whether a subject can be administered an agent (e.g., an agonist, antagonist, peptidomimetic, protein, peptide, nucleic acid, small molecule or other drug candidate) to treat a disease or disorder associated with aberrant GAVE1 expression or activity. For example, such methods can be used to determine whether a subject can be treated effectively with a specific agent or class of agents (e.g., agents of a type that decrease GAVE1 activity). Thus, the instant invention provides methods for determining whether a subject can be treated effectively with an agent for a disorder associated with aberrant GAVE1 expression or activity in which a test sample is obtained and GAVE1 protein or nucleic acid is detected (e.g., wherein the presence of GAVE1 protein or nucleic acid is diagnostic for a subject that can be administered the agent to treat a disorder associated with aberrant GAVE1 expression or activity).

The methods of the invention also can be used to detect genetic lesions or mutations in a GAVE1 gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a GAVE1-protein, or the misexpression of the GAVE1 gene. For example, such genetic lesions or mutations can be detected by ascertaining the existence of at least one of: 1) a deletion of one or more nucleotides from a GAVE1 gene; 2) an addition of one or more nucleotides to a GAVE1 gene; 3) a substitution of one or more nucleotides of a GAVE1 gene; 4) a chromosomal rearrangement of a GAVE1 gene; 5) an alteration in the level of a messenger RNA transcript of a GAVE1 gene; 6) an aberrant modification of a GAVE1 gene, such as of the methylation pattern of the genomic DNA; 7) the presence of a non-wild-type splicing pattern of a messenger RNA transcript of a GAVE1 gene; 8) a non-wild-type level of a GAVE1-protein; 9) an allelic loss of a GAVE1 gene; and 10) an inappropriate post-translational modification of a GAVE1 protein. As described herein, there are a large number of assay techniques known in the art that can be used for detecting lesions in a GAVE1 gene. A preferred biological sample is a peripheral blood leukocyte sample isolated by conventional means from a subject.

In certain embodiments, detection of the lesion involves the use of a probe/primer in a polymerase chain reaction (PCR) (See, e.g., U.S. Pat. Nos. 4,683,195 and 4,683,202), such as anchor PCR or RACE PCR, or, alternatively, in a ligation chain reaction (LCR) (See, e.g., Landegran et al., Science (1988) 241:1077-1080; and Nakazawa et al., Proc Natl Acad Sci USA (1994) 91:360-364), the latter of which can be particularly useful for detecting point mutations in the GAVE1 gene (See, e.g., Abravaya et al., Nucleic Acids Res (1995) 23:675-682). The method can include the steps of collecting a sample of cells from a patient, isolating nucleic acid (e.g., genomic, mRNA or both) from the cells of the sample, contacting the nucleic acid sample with one or more primers which specifically hybridize to a GAVE1 gene under conditions such that hybridization and amplification of the GAVE1 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., Proc Natl Acad Sci USA (1990) 87:1874-1878), transcriptional amplification system (Kwoh et al., Proc Natl Acad Sci USA (1989) 86:1173-1177), Q-β Replicase (Lizardi et al., Bio/Technology (1988) 6:1197), or any other nucleic acid amplification method, followed by the detection of the amplified molecules using techniques well known to those of skill in the art. The detection schemes are especially useful for the detection of nucleic acid molecules if such molecules are present in very low numbers.

In an alternative embodiment, mutations in a GAVE1 gene from a sample cell can be identified by alterations in restriction enzyme cleavage patterns. For example, sample and control DNA are isolated, amplified (optionally), digested with one or more restriction endonucleases, and fragment length sizes are determined by gel electrophoresis and compared. Differences in fragment length sizes between sample and control DNA indicate mutations in the sample DNA. Moreover, the use of sequence-specific ribozymes (see, e.g., U.S. Pat. No. 5,498,531) can be used to score for the presence of specific mutations by development or loss of a ribozyme cleavage site.

In other embodiments, genetic mutations in GAVE1 can be identified by hybridizing a sample and control nucleic acids, e.g., DNA or RNA, to high density arrays containing hundreds or thousands of oligonucleotides probes (Cronin et al., Human Mutation (1996) 7:244-255; Kozal et al., Nature Medicine (1996) 2:753-759). For example, genetic mutations in GAVE1 can be identified in two-dimensional arrays containing light-generated DNA probes as described in Cronin et al., supra. Briefly, a first hybridization array of probes can be used to scan through long stretches of DNA in a sample and to identify base changes between the sequences by making linear arrays of sequential overlapping probes. That step allows the identification of point mutations. That step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GAVE1 gene and to detect mutations by comparing the sequence of the sample GAVE1 with the corresponding wild-type (control) sequence. Examples of sequencing reactions include those based on techniques developed by Maxim & Gilbert (Proc Natl Acad Sci USA (1977) 74:560) or Sanger (Proc Natl Acad Sci USA (1977) 74:5463). It also is contemplated that any of a variety of automated sequencing procedures can be utilized when performing the diagnostic assays (Bio/Techniques (1995) 19:448), including sequencing by mass spectrometry (See, e.g., PCT Publication No. WO 94/16101; Cohen et al., Adv Chromatogr (1996) 36:127-162; and Griffin et al., Appl Biochem Biotechnol (1993) 38:147-159).

Other methods for detecting mutations in the GAVE1 gene include methods in which protection from cleavage agents is used to detect mismatched bases in RNA/RNA or RNA/DNA heteroduplexes (Myers et al., Science (1985) 230:1242). In general, the technique of “mismatch cleavage” entails providing heteroduplexes formed by hybridizing (labeled) RNA or DNA containing the wild-type GAVE1 sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine to digest mismatched regions. After digestion of the mismatched regions, the resulting material then is separated by size on denaturing polyacrylamide gels to determine the site of mutation. See, e.g., Cotton et al., Proc Natl Acad Sci USA (1988) 85:4397; Saleeba et al., Methods Enzymol (1992) 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GAVE1 cDNAs obtained from samples of cells. For example, the mutY enzyme of E. coli cleaves A at G/A mismatches and the thymidine DNA glycosylase from HeLa cells cleaves T at G/T mismatches (Hsu et al., Carcinogenesis (1994) 15:1657-1662). According to an exemplary embodiment, a probe based on a GAVE1 sequence, e.g., a wild-type GAVE1 sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in GAVE1 genes. For example, single strand conformation polymorphism (SSCP) may be used to detect differences in electrophoretic mobility between mutant and wild-type nucleic acids (Orita et al., Proc Natl Acad Sci USA (1989) 86:2766; See also Cotton, Mutat Res (1993) 285:125-144; Hayashi, Genet Anal Tech Appl (1992) 9:73-79). Single-stranded DNA fragments of sample and control GAVE1 nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al., Trends Genet (1991) 7:5).

In yet another embodiment, the movement of mutant or wild-type fragments in polyacrylamide gels containing a gradient of denaturant is assayed using denaturing gradient gel electrophoresis (DGGE) (Myers et al., Nature (1985) 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., Biophys Chem (1987) 265:12753).

Examples of other techniques for detecting point mutations include, but are not limited to, selective oligonucleotide hybridization, selective amplification or selective primer extension. For example, oligonucleotide primers may be prepared in which the known mutation is placed centrally and then hybridized to target DNA under conditions which permit hybridization only if a perfect match is found (Saiki et al., Nature (1986) 324:163); Saiki et al., Proc Natl Acad Sci USA (1989) 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al., Nucleic Acids Res (1989) 17:2437-2448) or at the extreme 3′ end of one primer where, under appropriate conditions, mismatch can prevent or reduce polymerase extension (Prossner, Tibtech (1993) 11:238). In addition, it may be desirable to introduce a novel restriction site in the region of the mutation to create cleavage-based detection (Gasparini et al., Mol Cell Probes (1992) 6:1). It is anticipated that in certain embodiments amplification also may be performed using Taq ligase for amplification (Barany, Proc Natl Acad Sci USA (1991) 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be used conveniently, e.g., in clinical settings to diagnose patients exhibiting symptoms or family history of a disease or illness involving a GAVE1 gene.

Furthermore, any cell type or tissue where GAVE1 is expressed may be utilized in the prognostic assays described herein.

### 3. Pharmacogenomics

Agents, or modulators which have a stimulatory or inhibitory effect on GAVE1 activity (e.g., GAVE1 gene expression) as identified by a screening assay described herein can be administered to individuals to treat (prophylactically or therapeutically) disorders (e.g., ischemic heart failure, ischemic reperfusion injury, inflammation, restenosis, immune disorders, dilated cardiomyopathy, apoptosis, to also include cardiomyocyte apoptosis, atherosclerosis and stroke) associated with GAVE1 activity. In conjunction with such treatment, the pharmacogenomics (i.e., the study of the relationship between the genotype of an individual and the response of that individual to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the genotype of an individual. Such pharmacogenomics further can be used to determine appropriate dosages and therapeutic regimens. Accordingly, the activity of GAVE1 protein, expression of GAVE1 nucleic acid or mutation content of GAVE1 genes in an individual can be determined thereby to select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder, Clin Chem (1997) 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” The pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes, CYP2D6 and CYP2C19) has provided an explanation as to why some patients do not obtain the expected drug effects or show exaggerated drug response and serious toxicity after taking the standard and safe dose of a drug. The polymorphisms are expressed in two phenotypes in the population, the extensive metabolizer (EM) and the poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by the CYP2D6-formed metabolite, morphine. The other extreme is the so called ultra-rapid metabolizers who do not respond to standard doses. Recently, the molecular basis of ultra-rapid metabolism has been identified to be due to CYP2D6 gene amplification.

Thus, the activity of GAVE1 protein, expression of GAVE1 nucleic acid or mutation content of GAVE1 genes in an individual can be determined to thereby select appropriate agent(s) for therapeutic or prophylactic treatment of the individual. In addition, pharmacogenetic studies can be used to apply genotyping of polymorphic alleles encoding the drug-metabolizing enzymes to the identification the drug responsiveness phenotype of an individual. That knowledge, when applied to dosing or drug selection, can avoid adverse reactions or therapeutic failure and thus enhance therapeutic or prophylactic efficiency when treating a subject with a GAVE1 modulator, such as a modulator identified by one of the exemplary screening assays described herein.

### 4. Monitoring of Effects During Clinical Trials

Monitoring the influence of agents (e.g., drugs, compounds) on the expression or activity of GAVE1 (e.g., the ability to modulate aberrant cell proliferation and/or differentiation) can be applied not only in basic drug screening, but also in clinical trials. For example, the effectiveness of an agent, as determined by a screening assay as described herein, to increase GAVE1 gene expression, protein levels or protein activity, can be monitored in clinical trials of subjects exhibiting decreased GAVE1 gene expression, protein levels or protein activity. Alternatively, the effectiveness of an agent, as determined by a screening assay, to decrease GAVE1 gene expression, protein levels or protein activity, can be monitored in clinical trials of subjects exhibiting increased GAVE1 gene expression, protein levels or protein activity. In such clinical trials, GAVE1 expression or activity and preferably, that of other genes that have been implicated in, for example, a cellular proliferation disorder, can be used as a marker of the immune responsiveness of a particular cell.

For example, and not by way of limitation, genes, including GAVE1, that are modulated in cells by treatment with an agent (e.g., compound, drug or small molecule) which modulates GAVE1 activity (e.g., as identified in a screening assay described herein) can be identified. Thus, to study the effect of agents on cellular proliferation disorders, for example, in a clinical trial, cells can be isolated and RNA prepared and analyzed for the levels of expression of GAVE1 and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of GAVE1 or other genes. In that way, the gene expression pattern can serve as a marker, indicative of the physiological response of the cells to the agent. Accordingly, the response state may be determined before, and at various points during, treatment of the individual with the agent.

In a preferred embodiment, the instant invention provides a method for monitoring the effectiveness of treatment of a subject with an agent (e.g., an agonist, antagonist, peptidomimetic, protein, peptide, nucleic acid, small molecule or other drug candidate identified by the screening assays described herein) comprising the steps of: (i) obtaining a pre-administration sample from a subject prior to administration of the agent; (ii) detecting the level of expression of a GAVE1 protein, mRNA or genomic DNA in the preadministration sample; (iii) obtaining one or more post-administration samples from the subject; (iv) detecting the level of expression or activity of the GAVE1 protein, mRNA or genomic DNA in the post-administration samples; (v) comparing the level of expression or activity of the GAVE1 protein, mRNA or genomic DNA in the pre-administration sample with the GAVE1 protein, mRNA or genomic DNA in the post-administration sample or samples; and (vi) altering the administration of the agent to the subject accordingly. For example, increased administration of the agent may be desirable to increase the expression or activity of GAVE1 to higher levels than detected, i.e., to increase the effectiveness of the agent. Alternatively, decreased administration of the agent may be desirable to decrease expression or activity of GAVE1 to lower levels than detected, i.e., to decrease the effectiveness of the agent.

### C. Methods of Treatment

The instant invention provides for both prophylactic and therapeutic methods of treating a subject at risk of (or susceptible to) a disorder or having a disorder associated with aberrant GAVE1 expression or activity. Such disorders include ischemic heart failure, ischemic reperfusion injury, restenosis, dilated cardiomyopathy, apoptosis, to also include cardiomyocyte apoptosis, atherosclerosis, immune system perturbations and stroke.

### 1. Prophylactic Methods

In one aspect, the invention provides a method for preventing in a subject, a disease or condition associated with an aberrant GAVE1 expression or activity, by administering to the subject an agent that modulates GAVE1 expression or at least one GAVE1 activity. Subjects at risk for a disease that is caused or contributed to by aberrant GAVE1 expression or activity can be identified by, for example, any or a combination of diagnostic or prognostic assays as described herein. Administration of a prophylactic agent can occur prior to the manifestation of symptoms characteristic of the GAVE1 aberrancy, such that a disease or disorder is prevented or, alternatively, delayed in progression. Depending on the type of GAVE1 aberrancy, for example, a GAVE1 agonist or GAVE1 antagonist agent can be used for treating the subject. The appropriate agent can be determined based on screening assays described herein.

### 2. Therapeutic Methods

Another aspect of the invention pertains to methods of modulating GAVE1 expression or activity for therapeutic purposes. The modulatory method of the invention involves contacting a cell with an agent that modulates one or more of the activities of GAVE1 protein activity associated with the cell. An agent that modulates GAVE1 protein activity can be an agent as described herein, such as a nucleic acid or a protein, a naturally-occurring cognate ligand of a GAVE1 protein, a peptide, a GAVE1 peptidomimetic or other small molecule. The agent can be a agonist, inverse agonist or antagonist. In one embodiment, the agent stimulates one or more of the biological activities of GAVE1 protein. Examples of such stimulatory agents include active GAVE1 protein and a nucleic acid molecule encoding GAVE1 that has been introduced into the cell. In another embodiment, the agent inhibits one or more of the biological activities of GAVE1 protein. Examples of such inhibitory agents include antisense GAVE1 nucleic acid molecules and anti-GAVE1 antibodies. The modulatory methods can be performed in vitro (e.g., by culturing the cell with the agent) or, alternatively, in vivo (e.g., by administering the agent to a subject). As such, the instant invention provides methods of treating an individual afflicted with a disease or disorder characterized by aberrant expression or activity of a GAVE1 protein or nucleic acid molecule. In one embodiment, the method involves administering an agent (e.g., an agent identified by a screening assay described herein) or combination of agents that modulates (e.g., upregulates or downregulates) GAVE1 expression or activity. In another embodiment, the method involves administering a GAVE1 protein or nucleic acid molecule as therapy to compensate for reduced or aberrant GAVE1 expression or activity.

Stimulation of GAVE1 activity is desirable in situations in which GAVE1 is abnormally downregulated and/or in which increased GAVE1 activity is likely to have a beneficial effect. Conversely, inhibition of GAVE1 activity is desirable in situations in which GAVE1 is abnormally upregulated and/or in which decreased GAVE1 activity is likely to have a beneficial effect.

The invention is further illustrated by the following examples which should not be construed as limiting. The contents of all references, patents and published patent applications cited throughout the instant application hereby are incorporated by reference.

### Example 1

### Cloning hGAVE1

PCR screening is performed on Marathon-Ready human heart cDNA (Clontech, Cat. No.7404-i) and human placenta cDNA (Clontech, Catalog No. 7411-i) with the cDNAs at a concentration of 0.5 ng/μl. Primers used for PCR are designed using the published human Edg-1 sequence (GenBank/EMBL accession number M31210) with the following sequences:

| Forward: | |
|---|---|
| 5′-GCGTTCGTCTGGAGTAGCGCCACC-3′ | (SEQ ID NO:7) |
| Nested Forward: | (SEQ ID NO:8) |
| 5′-GCGCCCAAGCTTGGCACCATGGGGCCCACCAGC-3′ | |
| Reverse: | |
| 5′-GCGACCAAGTAAAGAGCGCTTCCG-3′ | (SEQ ID NO:9) |
| Nested Reverse: | |
| 5′-GGGGGCCTCGAGCTAGGAAGAAGAG-3′ | (SEQ ID NO:10) |

The nested forward primer contains a HindIII restriction enzyme site followed by a Kozak sequence. The nested reverse primer contains an XhoI restriction enzyme site. The PCR is carried out in a Biometra Trio-Thermoblock thermocycler, using Pfu DNA polymerase (Stratagene) which is added to the PCR reaction following addition of template cDNA, primers, Pfu buffer and dNTP. The 50 μl reaction contains: 5 μl of 10×Pfu DNA buffer, 2 μl (2500 units/ml) of Pfu DNA polymerase, 1.0 μl of NTP mixture (containing 10 mM of each nucleotide); 2.0 μl of forward primer (10 mM); 2.0 μl of reverse primer (10 mM); 5 μl cDNA template and 33 μl of sterile water. The following cycles are used in the thermocycler: 94° C. for 2 minutes, followed by 30 cycles of 94° C. for 45 seconds, 58° C. for 45 seconds, 72° C. for 3 minutes and 72° C. for 10 minutes; followed by cooling down at 4° C.

Following PCR, 3 μl of dNTP (10 mM of each nucleotide) Clontech Catalog No. 7404-i and 1 μl (5 units) of Taq DNA polymerase (Qiagen, Catalog No. 201223) are added to the PCR product and incubated at 72° C. for 10 minutes. The PCR product then was run on a 1% agarose gel. A 1.1 kilobase band containing the desired fragment is cut from the gel and purified using the Qiaquick Gel Extraction Kit using the protocol provided by the manufacturer (Qiagen, Catalog No. 28704). The purified PCR product then is subcloned into a pCR2.1 vector (Invitrogen, Catalog No. K2000-01/40/J10 and K 2030-01/40/J10). To subclone the PCR product into the pCR2.1 vector, a ligation reaction is prepared using an Invitrogen TA Cloning vector kit. The ligation reaction contained: 5 μl sterile water; 1 μl Invitrogen 2× ligation buffer; 2 μl pCR2.1 vector (25 ng/μl ); 4 μl PCR product DNA (10 ng); 4 μl (5×) dilution buffer; and 1 μl T4 DNA ligase (5 units). The reaction is incubated for 18 hours at 14° C. E. coli cells are transformed with the ligation reaction by mixing 2 μl of the ligation reaction mixture with 200 μl of INValpha F′ competent E. coli cells (Invitrogen Catalog No. C658-00), incubation on ice for 30 minutes, heat shock at 370° C. for 45 seconds and incubation on ice for 2 minutes followed by addition of 800 μl of LB. The cells then are incubated overnight at 37° C. with agitation in a bacterial shaker/incubator (air is re-circulated). Following the overnight incubation, 200 μl of the transformation reaction mixture is plated onto LB agar plates containing 100 μg/ml ampicillin and incubated overnight at 37° C.

Following the incubation, 5 colonies were picked and each individual colony was grown in a separate tube overnight in 500 μl of LB containing 100 μg/ml ampicillin in a shaker/incubator. To screen colonies by PCR, the following reaction was used: 41.5 μl of a colony in LB; 5 μl Taq buffer (10×); 1.0 μl dNTP (10 mM of each nucleotide); 1.0 μl forward primer (10 mM); 1.0 μl reverse primer (10 mM); and 0.5 μl Taq DNA polymerase (5 units/μl).

The reaction was incubated in a Biometra Trio-Thermocycler using the following cycles: 94° C. for 2 minutes, 94° C. for 30 seconds, 55° C. for 30 seconds, 72° C. for 1 minute and 72° C. for 10 minutes followed by cooling down at 4° C.

To check the results of the PCR reaction, 5 μl of the PCR reaction is run on 1% TAB 1× agarose gel. Positive clones showed an insert of 1.145 kb. Positive clones are grown in 5 ml LB+100 μg/ml ampicillin overnight at 37° C. in a bacterial shaker/incubator. The plasmid is purified using a Qiagen DNA purification column as instructed in the manufacturer protocol (Qiagen Catalog No. 12143). The positive clones then are sequenced using a T7 forward primer (5′-TAATACGACTCACTATAGGG-3′) (SEQ ID NO: 11), a reverse primer (5′-CAGUAAACAGCTATGACCAT-3′) (SEQ ID NO: 12) and a gene-specific primer (5′-CCACCGACCCATGTAC-3′) (SEQ ID NO: 13). DNA sequencing identified isolation of a cDNA having the DNA sequence presented in FIG. 1 and the amino acid sequence presented in FIG. 2.

### Example 2

### Generation of CHO Cells Overexpressing hGAVE1

To provide significant quantities of hGAVE1 for further experiments, the cDNA encoding hGAVE1 is cloned into an expression vector and transfected into Chinese hamster ovary (CHO) cells.

To generate CHO expressing hGAVE1, CHO cells are plated in a six-well 35 mm tissue culture plate (3×105 CHO cells per well (ATCC Catalog No. CCL-61)) (Costar Catalog No.3516) in 2 ml of F12 HAM 1× media (Gibco/BRL, Catalog No. 11765-054) in the presence of 10% fetal bovine serum (Gibco/BRL Catalog No. 1600-044).

The cells then are incubated at 37° C. in a CO₂ incubator until the cells are 50-80% confluent. The cloned cDNA nucleic acid sequence of hGAVE1 is inserted using the procedure described above in a pcDNA 3.1 cloning vector (Invitrogen, Catalog No. V790-20). Two μg of the DNA are diluted into 100 μl of serum-free F12 HAM 1× media. Separately, 25 μl of Lipofectamine Reagent (Life Technologies, Catalog No. 18324-020) is diluted into 100 μl of serum-free F12 HAM 1× media. The DNA solution and the Lipofectamine solution then are mixed gently and incubated at room temperature for 45 minutes to allow for the formation of DNA-lipid complexes.

The cells are rinsed once with 2 ml of serum-free F12 HAM 1× media. For each transfection (six transfections in a six-well plate), 0.8 ml of serum-free F12 HAM 1× media is added to the solution containing the DNA-lipid complexes (0.2 ml total volume) and mixed gently. The resulting mixture (hereinafter the “transfection mixture”) then is overlaid (0.8 ml +0.2 ml) onto the rinsed cells. No anti-bacterial reagents are added. The cells then are incubated with the lipid-DNA complexes for 16 hours at 37° C. in a CO₂ incubator to allow for transfection.

After the completion of the incubation period, 1 ml of F12 HAM 1× media containing 10% fetal bovine serum is overlaid onto the cells without first removing the transfection mixture. At 18 hours after transfection, the media overlaying the cells is aspirated. Cells then are washed with 1×PBS pH 2-4 (Gibco/BRL Catalog No. 10010-023) and PBS is replaced with F12 HAM 1× media containing 5% serum (“selective media”). At 72 hours after transfection, the cells are diluted ten-fold into the selective medium containing the antibacterial agent genetecin at 400 μg/ml (Life Technologies, Catalog No. 11811).

### Example 3

### Agonist Assay

To screen for agonists of human GAVE1, hGAVE1 is artificially coupled to a G_{q} signaling package. Activation of the Gq mechanism stimulates the release of Ca²⁺ from sarcoplasmic reticulum vesicles within the cell. The Ca²⁺ is released into the cytoplasm where it can be detected using Ca²⁺ chelating dyes. A Fluorometric Imaging Plate Reader or FLIPR® apparatus (Molecular Devices) is used to monitor any resulting changes in fluorescence. The activity of an agonist is reflected by an increase in fluorescence.

CHO-K1 cells expressing hGAVE1 are pre-engineered to express an indiscriminate form of G_{q} protein (G_{α16}). To prepare such cells, G_{α16}-coupled CHO cells are obtained (Molecular Devices LIVEWARE TM cells, Catalog No. RD-HGA16) and the protocol in Example 2 followed to facilitate expression of hGAVE1 in the cells.

The cells are maintained in log phase of growth at 37° C. and 5% CO₂ in F12 HAM media (Gibco/BRL, Catalog No. 11765-054) containing 10% fetal bovine serum, 100 IU/ml penicillin (Gibco/BRL, Catalog No. 15140-148), 100 μg/ml streptomycin (Catalog No. 15140-148, Gibco/BRL), 400 μg/ml genetic (G418) (Gibco/BRL, Catalog No. 10131-035) and 200 μg/ml zeocin (Invitrogen, Catalog No. R250-05). Twenty-four hours prior to an assay, 12,500 cells/well of the CHO-K1 cells are plated in 384 well clear-bottomed assay plates with a well volume of 50 μl (Greiner/Marsh, Catalog No. N58102) using a 96/384 Multidrop device (Labsystems, Type 832). The cells are incubated at 37° C. in humidified 5% CO₂ incubator (Forma Scientific CO₂ water-jacketed incubator Model 3110).

The following stock solutions are prepared: a 1 M stock solution of Hepes (pH 7.5) (Gibco/BRL, Catalog No. 15630-080); a 250 mM stock solution of probenicid (Sigma, Catalog No. P8761) made in 1 N NaOH; and a 1 mM stock solution of Fluo 4-AM Dye (Molecular Probes, Catalog No. F14202) made in DMSO (Sigma D2650). Reaction buffer is prepared with 1000 ml Hank's Balanced Salt Solution (Fisher/Mediatech, Catalog No. MT21023), 20 ml of the 1 M Hepes stock solution and 10 ml of the 250 mM probenicid stock solution. To prepare the loading buffer, 1.6 ml of the 1 mM Fluo 4-AM Dye stock solution is mixed with 0.32 ml of pluronic acid (Molecular Probes, Catalog No. P6866) and then mixed with 400 ml of the above reaction buffer and 4 ml of fetal bovine serum.

One hour prior to the assay, 50 μl of freshly-prepared loading buffer is added to each well of the 384 well plate using a 96/384 Multidrop device. The cells are incubated at 37° C. in a humidified incubator to maximize dye uptake. Immediately prior to the assay, the cells are washed 2 times with 90 μl of reaction buffer using a 384 EMBLA Cell Washer (Skatron; Model No.12386) with the aspiration head set at least 10 mm above the plate bottom, leaving 45 μl of buffer per well.

The CCD camera (Princeton Instruments) of the FLIPR® II (Molecular Devices) instrument is set at an f-stop of 2.0 and an exposure of 0.4 seconds. The camera is used to monitor cell plates for accuracy of dye loading.

A compound library containing possible agonists will be tested at 10 μM concentration in physiological salt buffer. Changes in fluorescence are measured for 10 seconds prior to compound addition. After the addition of the compound, fluorescence is measured every second for the first minute followed by exposures taken every six seconds for a total experimental analysis time of three minutes. Five μl aliquots of the 100 μM stock compound are added after the tenth scan, giving a final compound concentration on the cells of 10 μM. The maximum fluorescence changes for the first 80 scans is recorded as a measure of agonist activity and compared to the maximum fluorescence change induced by 10 μM ATP (Sigma A9062).

### Example 4

### Antagonist Assay

To screen for antagonists of human GAVE1, hGAVE1 is artificially coupled to a G_{q} system. As in Example 3, a FLIPR® apparatus is used to monitor any resulting changes in fluorescence. The activity of an antagonist is reflected by any decrease in fluorescence.

CHO-K1 cells expressing hGAVE1 are pre-engineered to express an indiscriminate form of G_{q} protein (G_{α16}), as described above in Example 3. The cells are maintained in log phase of growth at 37° C. and 5% CO₂ in F12 HAM 1× media (Gibco/BRL, Catalog No. 11765-054) containing 10% fetal bovine serum, 100 IU/ml penicillin (Gibco/BRL, Catalog No. 15140-148), 100 μg/ml streptomycin (Catalog No. 15140-148, Gibco/BRL), 400 μg/ml genetecin (G418) (Gibco/BRL, Catalog No. 10131-035) and 200 μg/ml zeocin (Invitrogen, Catalog No. R250-05). Twenty-four hours prior to the assay, 12,500 cells/well of the CHO-K1 cells are plated in 384 well clear-bottomed assay plates with a well volume of 50 μl (Greiner/Marsh, Catalog No. N58102) using a 96/384 Multidrop device. The cells are allowed to incubate at 37° C. in humidified 5% CO₂.

The following stock solutions will be prepared: a 1 M stock solution of Hepes (pH 7.5) (Gibco/BRL, Catalog No. 15630-080); a 250 mM stock solution of probenicid (Sigma, Catalog No. P8761) made in 1 N NaOH; a 1 mM stock solution of Fluo 4-AM Dye (Molecular Probes, Catalog No. F 14202) made in DMSO (Sigma D2650); and a 480 nM stock solution of SPP-1 (BioMol). Reaction buffer is prepared with 1000 ml Hank's Balanced Salt Solution (Fisher/Mediatech, Catalog No. MT21023), 20 ml of the 1 M Hepes stock solution, 10 ml of the 250 mM probenicid stock solution and 1 mM CaCl₂. To prepare the loading buffer, 80 μl of the 1 mM Fluo 4-AM Dye stock solution are mixed with 16 μl of pluronic acid (Molecular Probes, Catalog No. P6866) and then mixed with 20 ml of the above reaction buffer and 0.2 ml of fetal bovine serum.

Thirty minutes prior to the assay, 30 μl of freshly-prepared loading buffer are added to each well of the 384 well plate using a 96/384 Multidrop device. The cells are incubated at 37° C. in a humidified CO₂ incubator during that time to maximize dye uptake. Immediately prior to the assay, the cells are washed 3 times with 100 μl of reaction buffer using a 384 EMBLA Cell Washer with the aspiration head set at least 40 mm above the plate bottom, leaving 45 μl of buffer per well.

Five μl of the 100 μM stock compound are added to the cells using a Platemate-384 pipetor (Matrix). The compound concentration during the incubation step will be approximately 10 μM. The cells then are placed on the FLIPR® II and plate fluorescence is measured every second for the first minute followed by exposures taken every six seconds for a total experimental analysis time of three minutes. Sphingosine-1-phosphate (10 μM, BioMol SL 140) is added after the tenth scan. After each addition, the pipet tips are washed 10 times with 20 μl of 0.01% DMSO in water.

### Example 5

### Receptor Binding Assay

To prepare membrane fractions containing hGAVE1 receptor, CHO cell lines overexpressing hGAVE1 are harvested by incubation in phosphate-buffered saline (10 ml) containing 1 mM EDTA. The cells are washed further 3 times in phosphate-buffered saline containing 1 mM EDTA (10 ml) prior to resuspension in 5 ml of Buffer A (50 mM Tris-HCl (pH 7.8) (Sigma T6791), 5 mM MgCl₂ (Sigma M8266) and 1 mM EGTA (Sigma 0396).

The cells then are disrupted with a tissue homogenizer (Polytron, Kinemetica, Model PT 10/35) for 1 minute. The resulting homogenate is centrifuged in a Sorvall Instruments RC3B refrigerated centrifuge at 49,000×g at 4° C. for 20 minutes. The resulting pellet is resuspended in 25 ml of Buffer A and the centrifugation step is repeated three times. Following the final centrifugation, the pellet again is resuspended in 5 ml of Buffer A, aliquoted and stored at −70° C.

A receptor binding assay using the membrane fraction and radiolabeled sphingosine-1-phosphate (SPP-1) as a tracer is performed. The assay is performed in a 96 well plate (Beckman Instruments). The binding reaction consisted of 18 μg of the CHO cell preparation in the presence of radioactive SPP-1 (0.01 nM-25 nM) in a final volume of 0.2 ml of Buffer A containing 0.1% bovine serum albumin (Sigma, Catalog No. 34287) (See Im et al., J Biol Chem (2000) 275(19):14281-14286). The reaction is incubated for 1 hour at room temperature. The reaction is terminated by filtration through Whatman GF/C filters on a multichannel harvester (Brandell) which is pretreated with 0.3% polyethyleneimine (Sigma, Catalog No. P3143) and 0.1% bovine serum albumin (BSA) for 1 hour. The mixture is applied to the filter and incubated for one hour. The filters are washed 6 times with 1 ml of ice cold 50 mM Tris-HCl pH 7.6. Specific binding is calculated based on the difference between total binding and non-specific binding (background) for each tracer concentration by measuring the radioactivity. Eight to 16 concentration data points are needed to determine the binding of ligand to the receptor achieved in an equilibrium state between the ligand and receptor (equilibrium binding parameters) and the amount of nonradioactive SPP-1 needed to compete for the binding of radioactive SPP-1 on the receptor (competition binding values). Inhibition curves are prepared to determine the concentration required to achieve a 50% inhibition of binding (IC₅₀).

### Example 6

### Northern Blot Analysis

Northern blot analysis is performed on RNA derived from several human tissue samples to determine whether the tissues express the hGAVE1 receptor gene. The probe used is P³²-labeled hGAVE1 cDNA.

### Preparation of the Probe

P32-labeled hGAVE1 cDNA is prepared as follows. Twenty-five ng hGAVE1 cDNA prepared as described above is resuspended to 45 μl of 10 mM Tris-HCl, pH 7.5; 1 mM EDTA in a microfuge tube and heated at 95° C. for 5 minutes. The tube then is chilled on ice for another 5 minutes. Following chilling, the mixture contained in the tube is resuspended with the 45 μl GAVE1 cDNA and buffer as described above and mixed with RTS Rad Prime Mix (supplied with the RTS Rad Prime DNA-labeling System) (Life Technologies, Catalog No. 10387-017). Five μl of P³²-labeled α-dCTP, specific activity: 3000 Ci/mM, (Amersham, AA0005) is added while mixing gently but thoroughly. The resulting mixture is incubated at 37° C. for 10 minutes. Incubation is stopped by the addition of 5 μl of 0.2 M EDTA, pH 8.0. Incorporation of the radioactive alpha-dCTP into the hGAVE1 cDNA is evaluated by taking a 5 μl aliquot of the mixture and counting the radioactivity.

### Preparation of the Cell Cultures

Cell cultures of cardiac myocytes from 1 day-old rats are prepared following the procedure described in Argentin et al. (Mol Cell Biol (1997) 14(1):777-790).

### RNA Extraction

Primary cardiac myocyte cells were stimulated for 48 hours with 10⁻⁵ M phenylephrine as described in Ardati et al. (EMBO J (1993) 12:5131-5139). Following stimulation, the cells are directly lysed in a culture dish by adding 1 ml of Trizol Reagent (Life Technologies, Catalog No. 15596). The cell lysate then is passed through a pipette several times to homogenize the lysate (cell lysate is subsequently is transferred to a tube). Following homogenization, the lysate is incubated for 5 minutes at 30 ° C. to permit the complete dissociation of nucleoprotein complexes. Following incubation, 0.2 ml of chloroform (Sigma, Catalog No. C53 12) per 1 ml of Trizol Reagent is added to the lysate and the tube is shaken vigorously for 15 seconds. The lysate then is incubated at 30° C. for 3 minutes. Following incubation, the lysate is centrifuged at 12,000×g for 15 minutes at 4° C. The resulting aqueous phase is transferred to a fresh tube and 0.5 ml of isopropyl alcohol per 1 ml of Trizol Reagent is added. The aqueous phase sample then is incubated at 30° C. for 10 minutes and centrifuged at 12,000×g for 10 minutes at 4° C. Following centrifugation, the supernatant is removed and the remaining RNA pellet is rinsed with 70% ethanol.

The rinsed sample then is centrifuged at 7500×g for 10 minutes at 40° C. and the resulting supernatant is discarded. The remaining RNA pellet then is dried and resuspended in RNase-free water (Life Technologies, Catalog No. 10977-015).

### Gel Electrophoresis

An agarose gel is prepared by melting 2 g of agarose (Sigma, Catalog No. A0169) in water, 5× formaldehyde gel-running buffer (See below for description) and 2.2 M formaldehyde (Sigma, Catalog No. P82031).

Samples for gel electrophoresis are prepared as follows:

RNA 4.5 μl (5 μg total)

5× formaldehyde gel-running buffer 2.0 μl

formaldehyde 3.5 μl

formamide (Sigma, Catalog No. F9037) 10.0 μl

(5× formaldehyde gel-running buffer is 0.1M 3-(N-morpholino) propanesulfonic acid (MOPS) (pH 7.0) (Sigma, Catalog No. M5162); 40 mM sodium acetate (Sigma, Catalog No. S7670); and 5 mM EDTA (pH 8.0) (Sigma, Catalog No. E7889)).

The samples are incubated for 15 minutes at 65° C. and then chilled on ice. After chilling, the samples are centrifuged for 5 seconds. Two μl of formaldehyde gel-loading buffer; 50% glycerol (Sigma, Catalog No. G5516); 1 mM EDTA (pH 8.0); 0.25% bromophenol blue (Sigma, Catalog No. 18046); and 0.25% xylene cyanol FF (Sigma, Catalog No. 335940) then are added to the sample.

Alternatively, commercially available (Clontech) hybridization filters containing a plurality of RNA samples from various organs can be purchased.

The gel is pre-run for 5 minutes at 5 V/cm. Following the pre-run, the samples are loaded onto the gel. The gel then is run at 4 V/cm while submerged in 1x formaldehyde gel-running buffer. The buffer is changed at 2 hours into the run.

### Transfer of RNA from Gel to Nitrocellulose

The gel is stained with ethidium bromide (Sigma, Catalog No. El 385) (0.5 μg/ml in 0.1M ammonium acetate (Sigma, Catalog No. 09689)) for 30 minutes to insure that RNA is not degraded. The RNA then is transferred from the agarose gel to a nitrocellulose filter (Shleicher & Schuell Inc, Catalog No. 74330-026) using the protocol described in Sambrook et al., eds. (in Molecular Cloning: A Laboratory Manual, volume 1, pp.7.46-7.51, Cold Spring Harbor Laboratory Press (1989)).

### Hybridization of P³²-labeled cDNA

Clontech ExpressHyb hybridization solution (Clontech, Catalog No. 8015-1) is incubated at 68° C. for 2 hours. Following incubation, 15 ml of the warmed hybridization solution is poured onto the multiple tissue sample Northern (MTN) membrane. The MTN membrane is left soaking in the hybridization solution at 68° C. while shaking. After 1 hour has elapsed, the hGAVE1 cDNA probe, which had been previously denatured by boiling at 95° C. for 5 minutes, is added at a concentration of 10⁶ counts/ml. The incubation of the hybridization solution covering the gel at 68° C. then is continued for 2 hours while shaking.

The MTN membrane then is removed from the Clontech ExpressRyb hybridization solution and is washed 3 consecutive times with Clontech Wash Solution 1(2× SSC; 0.05% SDS) by dipping the membrane into 15 ml of solution while shaking at room temperature for 40 minutes, respectively, with solution changes every 40 minutes. Clontech Wash Solution 2 (0.1× SSC; 0.1% SDS) then is warmed at 55° C. for 1 hour. The membrane then is washed 3 consecutive times with Clontech Wash Solution 2 (0.1× SSC; 0.1% SDS) by dipping the membrane into 15 ml of solution while shaking at 55° C. temperature for 60 minutes. The wash solution is changed every 15 minutes.

### Development

The membrane is exposed to Kodak X-OMAT AR (Kodak, Catalog No. 165 1579) film overnight at −70° C.

The Northern protocol for the rat cardiomyocytes is performed as outlined in Argentin et al. Mol Cell Biol (1994) 14:770-790.

### Results

hGAVE1 is expressed at high levels in the human brain, heart, spleen and lung. GAVE1 also is expressed in the intestine, skeletal muscle and placenta. Lower levels of hGAVE1 transcript are detected in the liver and kidney. The size of the unique transcript is about 2.2 kb. Endothelial cells express GAVE1. S-1-P is a ligand of GAVE1.

The level of GAVE1 expression in phenylephrine (PE)-treated rat cardiac myocytes is up-regulated several-fold as compared to that in control (untreated) or vehicle-treated rat cardiac myocytes. PE is an α-1-adrenergic agonist known to cause cardiac hypertrophy in vitro. The hypertrophy is characterized by morphologic changes, such as increased cell size and stronger contractility. Left untreated, cardiac hypertrophy leads to congestive heart failure. The fact that the GAVE1 receptor is modulated in cardiac hypertrophy models implicates the gene in disease/pathology states such as congestive heart failure.

### Example 7

### PCR Assay

TaqMan® or real time RT-PCR is a powerful tool for detecting messenger RNA in samples. The technology exploits the 5′ nuclease activity of AmpliTaq Gold® DNA polymerase to cleave a TaqMan® probe during PCR. The TaqMan® probe contains a reporter dye (in the experiments: 6-FAM (6-carboxyfluorescein)) at the 5′-end of the probe and a quencher dye (in the experiments: TAMRA (6-carboxy-N, N, N′, N′-tetramethylrhodamine)) at the 3′-end of the probe. TaqMan′ probes are designed specifically to hybridize with the target cDNA of interest between the forward and the reverse primer sites. When the probe is intact, the 3′-end quencher dye suppresses the fluorescence of the 5′-end reporter dye. During PCR, the 5′→3′ activity of the AmpliTaq Gold® DNA polymerase results in the cleavage of the probe between the 5′-end reporter dye and the 3′-end quencher dye resulting in the displacement of the reporter dye. Once displaced, the fluorescence of the reporter dye no longer is suppressed by the quencher dye. Thus, the accumulation of PCR products made from the targeted cDNA template is detected by monitoring the increase in fluorescence of the reporter dye.

An ABI Prism Sequence detector system from Perkin Elmer Applied Biosystems (Model No. ABI7700) is used to monitor the increase of the reporter fluorescence during PCR. The reporter signal is normalized to the emission of a passive reference.

### Preparation of cDNA Template

Total RNA and poly A⁺ RNA from several tissues are purchased from Clontech (See Tables 1 and 2 for catalog numbers).

TABLE 1

| Human Total RNA | Clontech Cat. No. |
|---|---|
| Human Brain, whole | 64020-1 |
| Human Heart | 64025-1 |
| Human Kidney | 64030-1 |
| Human Liver | 64022-1 |
| Human Lung | 64023-1 |
| Human Pancreas | 64031-1 |
| Human Skeletal Muscle | 64033-1 |
| Human Small Intestine | 64039-1 |
| Human Spleen | 64034-1 |
| Human Stomach | 64090-1 |
| Human Thymus | 64028-1 |

TABLE 2

| RNA Sample | Clontech Catalog No. |
|---|---|
| Human Brain, whole | 6516-1 |
| Human Brain, amygdala | 6574-1 |
| Human Brain, caudate nucleus | 6575-1 |
| Human Brain, cerebellum | 6543-1 |
| Human Brain, corpus collosum | 6577-1 |
| Human Brain, hippocampus | 6578-1 |
| Human Brain, substantia nigra | 6580-1 |
| Human Brain thalamus | 6582-1 |
| Human Fetal Brain | 6525-1 |

Five μg of total RNA is mixed with 2 μl (50 ng/μl) of random hexamer primers (Life Technologies, Catalog No. 18090) for a total reaction volume of 7 μl. The resulting mixture is heated at 70° C. for 10 minutes and quickly chilled on ice. The following then are added to the mixture: 4 μl of 5× first strand buffer, 2 μl of 0.1 mM DTT, 1 μl of 10 mM dNTP and 1 μl of water. The mixture is mixed gently and incubated at 37° C. for 2 minutes. Following the incubation, 5 μl of Superscript RT-PCR reverse transcriptase (Life Technologies, Catalog No. 18090) are added. The mixture then is incubated at 37° C. for 60 minutes. The reaction is stopped by the addition of 1 μl of 2.5 mM EDTA. The mixture then is incubated for 65° C. for 10 minutes.

The PCR and TaqMan® assay are performed in a 96 well plate MicroAmp optical tube (Perkin Elmer, Catalog No. N801-0933). A reaction mixture comprising 25 μl of TaqMan® PCR Mixture (Perkin Elmer, Catalog No. N808-0230), 1 μl forward primer (5′-TCTGCGGGAAGGGAGTATGT-3′) (SEQ ID NO: 14) 1 μl of reverse primer (5′-CGATGGCGAGGAGACTGAA-3′) (SEQ ID NO: 15) 1 μl of TaqMan® probe (5′-TGTGGCCCTGTCAGCCTCCGT-3′) (SEQ ID NO: 16) 1 μl cDNA, and 21 μl of water are placed into each well. TaqMan® samples are created in duplicate for each tissue sample at the following cDNA template concentrations: 5, 2, 1, 0.5, 0.25, 0.125, 0.0625 ng/μl (the template cDNA concentration is a final concentration). The plate then is sealed with MicroAmp optical 8-strip caps (Perkin Elmer, Catalog No. N801-0935).

A standard curve is performed in duplicate using the human β-actin gene (Perkin Elmer, Catalog No. 401846). For each cDNA template concentration of the standard curve, a number of amplified molecules are obtained. Having a standard curve amplification of a known gene allows for quantification of cDNA molecules amplified for each unknown target gene and normalization with an internal control.

Results from the above TaqMan® reactions are expressed relative to a tissue of arbitrary choice as fold regulation (for instance, value of the GAVE1 expression in the heart/value of the GAVE1 expression in the brain). For the central nervous system, cerebellum is chosen as a reference to which the expression of GAVE1 is compared to and relative fold expression is deduced.

Peripheral lymphocytes were separated into Th1 and Th2 populations. The Th1 and Th2 cells were transformed to express GAVE1. Resting cells expressed GAVE1. When cells were exposed to CD3 and CD28 antibodies that activate Th1 and Th2 cells, GAVE1 expression was inhibited. The inhibition was prolonged.

Although the instant invention has been described in detail with reference to the examples above, it is understood that various modifications can be made without departing from the spirit of the invention. Accordingly, the invention is limited only by the following claims.

All cited patents and publications referred to in this application are herein incorporated by reference in their entirety.

We claim:

1. An isolated nucleic acid comprising the nucleotide sequence of GAVE1 (SEQ ID NO: 1) or a variant of GAVE1, wherein said variant encodes amino acids comprising residues 250-252, residue 382 or residues 250-252 and 382 of SEQ ID NO: 2.

2. The isolated nucleic acid of claim 1, wherein said sequence encodes a GAVE1 polypeptide with the amino acid sequence of SEQ ID NO: 2.

3. The nucleic acid of claim 1, wherein said nucleic acid is selected from the group consisting of RNA, genomic DNA, synthetic DNA and cDNA.

4. An isolated nucleic acid encoding a GAVE1 fragment comprising residues 250-252, residue 382 or residues 250-252 and 382 of SEQ ID NO: 2.

5. The nucleic acid according to claim 2, encoding a substitution mutation that provides at least one functionally-equivalent amino acid residue.

6. An isolated nucleic acid comprising a sequence that hybridizes under stringent conditions to the nucleotide sequence of SEQ ID NO: 1 or the complement of SEQ ID NO: 1, and wherein said nucleic acid encodes amino acid residues 250-252, residue 382 or residues 250-252 and 382 of SEQ ID NO: 2.

7. A purified polypeptide comprising the third intracellular loop of SEQ ID NO: 2.

8. An expression vector comprising the nucleic acid of claim 1, operably linked to an expression control element.

9. The expression vector of claim 8, wherein said expression control element is selected from the group consisting of constitutive, cell-specific and inducible regulatory sequences.

10. An expression vector comprising a cDNA sequence encoding a nucleotide sequence that expresses GAVE1 of SEQ ID NO: 2 or a polypeptide comprising 250-252 and/or residue 382 as set forth in SEQ ID NO: 2.

11. A cultured cell comprising the vector of claim 8.

12. A cultured cell comprising the nucleic acid of claim 1 operably linked to an expression control element.

13. A cultured cell transformed with the vector of claim 8, or a progeny of said cell, wherein said cell expresses the polypeptide encoded by the nucleic acid comprising said vector.

14. The cultured cell of claim 11, wherein said cell is selected from the group consisting of eukaryotic cells and prokaryotic cells.

15. A method of producing a protein, comprising culturing the cell of claim 11 under conditions permitting expression of the polypeptide encoded by the nucleic acid of said vector.

16. An antibody that binds specifically to GAVE1.

17. The antibody of claim 16, which is a monoclonal antibody or a polyclonal antibody.

18. The antibody of claim 16, wherein said antibody prevents the binding of a ligand to GAVE1 .

19. A therapeutic method for modulating GAVE1 signaling activity or signal transduction in a patient in need of treatment comprising administering to said patient an agonist, an antagonist or an inverse agonist of GAVE1.

20. A method for identifying an agonist of GAVE1 comprising:
i) contacting a potential agonist with a cell expressing GAVE1; and
ii) determining whether in the presence of said potential agonist the signaling activity of GAVE1 is increased relative to the activity of GAVE1 in the absence of said potential agonist.

21. A method for identifying an inverse agonist to GAVE1 comprising:
i) contacting a potential inverse agonist with a cell expressing GAVE1; and
ii) determining whether in the presence of said potential inverse agonist, the activity of GAVE1 is decreased relative to the activity of GAVE1 in the absence of said potential inverse agonist, and in the absence of a ligand or an agonist.

22. The method of claim 21, wherein said ligand is sphingosine-1-phosphate.

23. A method for identifying an antagonist to GAVE1 comprising:
i) contacting a potential antagonist with a cell expressing GAVE1; and
ii) determining whether in the presence of said potential antagonist the signaling activity of GAVE1 is decreased relative to the activity of GAVE1 in the presence of a ligand or an agonist.

24. The method of claim 23, wherein said ligand is sphingosine-1 -phosphate.

25. A therapeutic composition comprising an agonist, an antagonist or an inverse agonist of GAVE1 capable of modulating GAVE1 signaling activity or transduction, and a pharmaceutically acceptable carrier, excipient or diluent.

26. A method for treating a disease comprising administering to a patient in need of treatment a therapeutic composition comprising an agonist, an antagonist or an inverse agonist of GAVE1 capable of modulating GAVE1 signaling activity or transduction, and a pharmaceutically acceptable carrier, excipient or diluent.
